(19) United States
(12) Patent Application Publication
(10) Pub. No.: US 2024/0038362 A1
(43) Pub. Date: Feb. 1, 2024

(54) METHODS AND APPARATUS FOR RADIOABLATION TREATMENT

(71) Applicants: VARIAN MEDICAL SYSTEMS, INC., Palo Alto, CA (US); SIEMENS HEALTHINEERS INTERNATIONAL AG, Steinhausen (CH)

(72) Inventors: Jonas Michael HONEGGER, Zurich (CH); Francesca ATTANASI, Baar (CH)

(21) Appl. No.: 18/267,972

(22) PCT Filed: Dec. 18, 2020
(86) PCT No.: PCT/US2020/066213

§ 371 Date: Jun. 16, 2023

(51) Int. Cl. G16H 20/40; G06T 17/00; G06T 19/20; G16H 10/60

(52) U.S. Cl. CPC G16H 20/40; G06T 17/00; G06T 19/20; G16H 10/60; G06T 2219/2004; G06T 2210/41; G06T 2200/24

(57) ABSTRACT

Systems and methods for radioablation treatment planning are disclosed. In some examples, a computing device provides for display a user interface that allows a medical professional to define a target region of a patient for treatment. The user interface may allow the medical professional to select a treatment area using interactive target maps generated for the patient. The computing device also receives image data from an imaging system for the patient, such as image data identifying a 3D volume of the patient's scanned structure. The computing device may generate for display a 3D image of the scanned structure based on the received image data, and may superimpose on the 3D image a target region map that the medical professional can manipulate to define the target region of treatment for the patient. Once defined, the computing device may transmit the defined target region to a treatment system for treating the patient.

### FIELD

Aspects of the present disclosure relate in general to medical diagnostic and treatment systems and, more particularly, to providing radioablation diagnostic, treatment planning, and delivery systems for diagnosis and treatment of conditions, such as cardiac arrhythmias.

### BACKGROUND

Various technologies can be employed to capture or image a patient's metabolic, electrical and anatomical information. For example, positron emission tomography (PET) is a metabolic imaging technology that produces tomographic images representing the distribution of positron emitting isotopes within a body. Computed Tomography (CT) and Magnetic Resonance Imaging (MRI) are anatomical imaging technologies that create images using x-rays and magnetic fields respectively. Images from these exemplary technologies can be combined with one another to generate composite anatomical and functional images. For example, software systems, such as Velocity™ software from Varian Medical Systems, Inc. combine different types of images using an image fusion process to deform and/or register images to produce a combined image.

In cardiac radioablation, medical professionals work together to diagnose cardiac arrhythmias, identify regions for ablation, prescribe radiation treatment, and create radioablation treatment plans. Typically, each of the various medical professionals have complementary medical training and thus specialize in varying aspects of the treatment development. For example, an electrophysiologist may identify one or more regions or targets of a patient's heart for treatment of cardiac arrhythmias based on a patient's anatomy and electrophysiology. The electrophysiologist may use, for example, combined PET and cardiac CT images as inputs to manually define a target region for ablation. Once a target region is defined by the electrophysiologist, a radiation oncologist may prescribe radiation treatment including, for example, the number of fractions of radiation to be delivered, radiation dose to be delivered to a target region and maximum dose to adjacent organs at risk. Once a radiation dose is prescribed, typically a dosimetrist may create a radioablation treatment plan based on the prescribed radiation therapy. The radiation oncologist then typically reviews and approves the treatment plan to be delivered. In addition, and prior to finalization of the radioablation treatment plan, the electrophysiologist may want to understand the location, size, and shape of a dose region of the defined target volume to confirm the target location for the patient as defined by the radioablation treatment plan is correct.

Properly identifying and defining the target region of a patient's organ for treatment is essential for developing and optimizing the treatment plan. For example, an over-inclusive target region may result in a defined target volume that includes areas that do not require treatment, while an under-inclusive target region may result in a defined target volume that fails to include areas that should be treated. As such, there are opportunities to improve radioablation treatment planning systems used by medical professionals, such as cardiac radioablation treatment systems used for cardiac radioablation diagnosis and radiation treatment planning.

### SUMMARY

Systems and methods for cardiac radioablation diagnosis treatment and planning are disclosed. In some examples, a computing device provides for display a user interface that allows a medical professional to define a target region of a patient for treatment. The user interface may allow the medical professional to select a treatment area using interactive target maps generated for the patient. The computing device also receives image data from an imaging system for the patient, such as image data identifying a 3D volume of the patient's scanned structure. The computing device may generate for display a 3D image of the scanned structure based on the received image data, and may superimpose on the 3D image a target region map that the medical professional can manipulate to define the target region of treatment for the patient. Once defined, the computing device may transmit the defined target region to a treatment system for treating the patient.

In some examples, a system includes a computing device that is configured to receive a first input identifying a treatment target area of an organ of a patient, and receive a scanned image of the organ. The computing device is also configured to generate a first digital model of a type of the organ. Further, the computing device is configured to determine an alignment of the scanned image to the first digital model. The computing device is also configured to generate a second digital model comprising at least a portion of the scanned image and the first digital model. The computing device is further configured to store the second digital model in a data repository.

In some examples, a computer-implemented method includes receiving a first input identifying a treatment target area of an organ of a patient, and receiving a scanned image of the organ. The method also includes generating a first digital model of a type of the organ. Further, the method includes determining an alignment of the scanned image to the first digital model. The method also includes generating a second digital model comprising at least a portion of the scanned image and the first digital model. The method further includes storing the second digital model in a data repository.

In some examples, a non-transitory computer readable medium storing instructions that, when executed by at least one processor, cause the at least one processor to perform operations including receiving a first input identifying a treatment target area of an organ of a patient, and receiving a scanned image of the organ. The operations also include generating a first digital model of a type of the organ. Further, the operations include determining an alignment of the scanned image to the first digital model. The operations also include generating a second digital model comprising at least a portion of the scanned image and the first digital model. The operations further include storing the second digital model in a data repository.

In some examples, a method includes a means for receiving a first input identifying a treatment target area of an organ of a patient, and receiving a scanned image of the organ. The method also includes a means for generating a first digital model of a type of the organ. Further, the method includes a means for determining an alignment of the scanned image to the first digital model. The method also includes a means for generating a second digital model comprising at least a portion of the scanned image and the first digital model. The method further includes a means for storing the second digital model in a data repository.

### BRIEF DESCRIPTION OF THE DRAWINGS

The features and advantages of the present disclosures will be more fully disclosed in, or rendered obvious by the following detailed descriptions of example embodiments. The detailed descriptions of the example embodiments are to be considered together with the accompanying drawings wherein like numbers refer to like parts and further wherein.

FIG. 1 illustrates a cardiac radioablation diagnosis and treatment system, in accordance with some embodiments;

FIG. 2 illustrates a block diagram of a target definition computing device, in accordance with some embodiments;

FIG. 3 illustrates exemplary portions of the cardiac radioablation treatment system of FIG. 1, in accordance with some embodiments;

FIGS. 4A, 4B, 4C, 4D, 4E, and 4F illustrate portions of a graphical user interface, in accordance with some embodiments;

FIGS. 5A and 5B illustrate portions of a graphical user interface, in accordance with some embodiments;

FIGS. 6A, 6B, 6C, 6D, 6E, and 6F illustrate portions of a graphical user interface, in accordance with some embodiments;

FIG. 7A illustrates a 2-dimensional segment model, in accordance with some embodiments;

FIG. 7B illustrates a 3-dimensional segment model, in accordance with some embodiments;

FIG. 7C illustrates a 3-dimensional segment model with a septum border, in accordance with some embodiments;

FIG. 8 illustrates editing options for the 2-dimensional segment model of FIG. 7A, in accordance with some embodiments;

FIG. 9 illustrates editing options for the 3-dimensional segment model of FIG. 7B, in accordance with some embodiments;

FIG. 10A illustrates the selection of a segment within a segment model, in accordance with some embodiments;

FIG. 10B illustrates a 3-dimensional segment model identifying a selected segment, in accordance with some embodiments;

FIG. 11 is a flowchart of an example method to generate a study for a patient, in accordance with some embodiments;

FIG. 12 is a flowchart of an example method to generate an interactive map for identifying a treatment target area, in accordance with some embodiments;

FIG. 13A is a flowchart of an example method to generate a digital model, in accordance with some embodiments; and

FIG. 13B is a flowchart of an example method to adjust an orientation of the digital model of FIG. 13A, in accordance with some embodiments.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

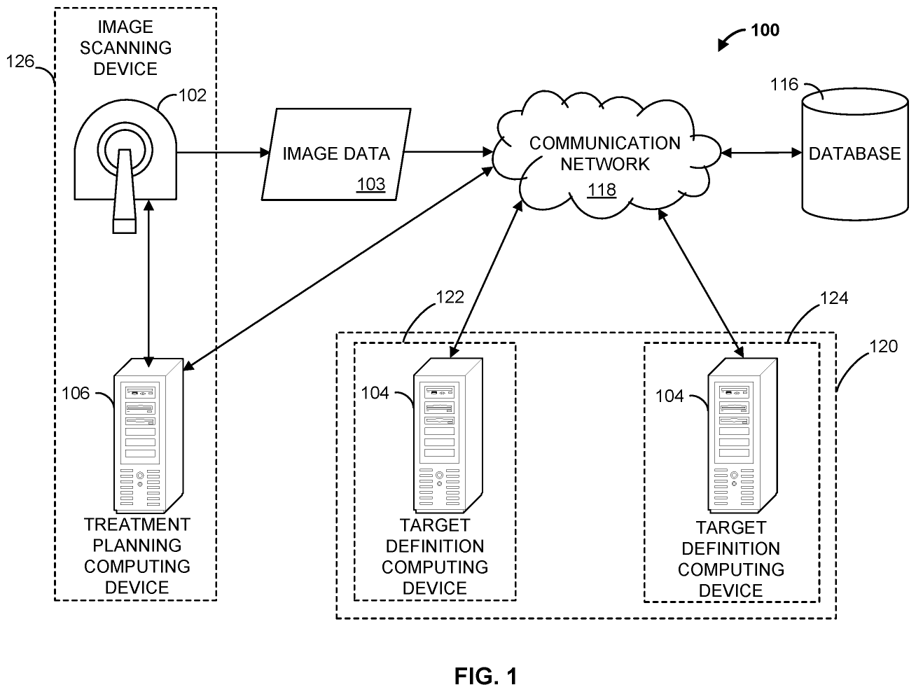

Turning to the drawings, FIG. 1 illustrates a block diagram of a cardiac radioablation diagnosis and treatment system 100 that includes an imaging device 102, a treatment planning computing device 106, one or more target definition computing devices 104, and a database 116 communicatively coupled over communication network 118. Imaging device 102 may be, for example, a CT scanner, an MR scanner, a PET scanner, an electrophysiologic imaging device, an ECG, or an ECG imager. In some examples, imaging device 102 may be PET/CT scanner or a PET/MR scanner. In some examples, imaging device 102 and treatment planning computing device 106 may be part of a radioablation treatment system 126 that allows for radioabaltion treatment to a patient. For example, radioablation treatment system 126 may allow for the delivery of defined doses to one or more treatment areas of the patient.

Each target definition computing device 104 and treatment planning computing device 106 can be any suitable computing device that includes any suitable hardware or hardware and software combination for processing data. For example, each can include one or more processors, one or more field-programmable gate arrays (FPGAs), one or more application-specific integrated circuits (ASICs), one or more state machines, digital circuitry, or any other suitable circuitry. In addition, each can transmit data to, and receive data from, communication network 118. For example, each of target definition computing device 104 and treatment planning computing device 106 can be a server such as a cloud-based server, a computer, a laptop, a mobile device, a workstation, or any other suitable computing device.

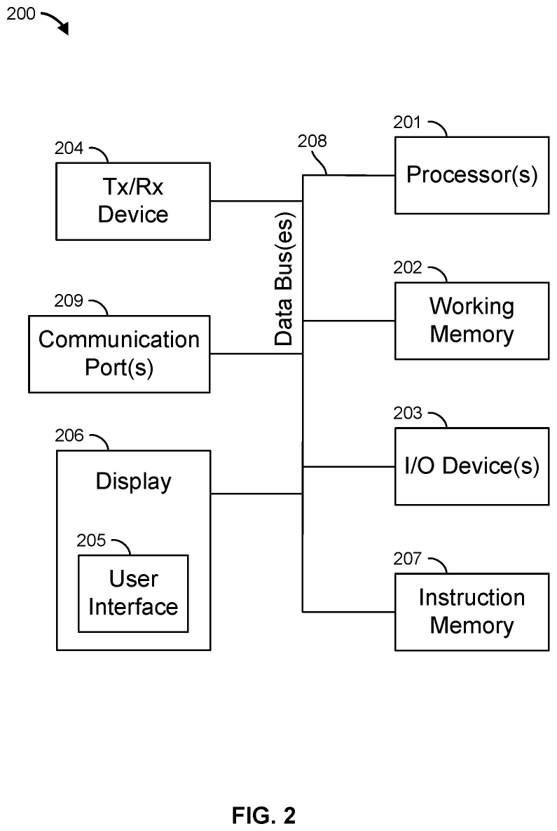

For example, FIG. 2 illustrates a computing device 200, which may be an example of each of target definition computing device 104 and treatment planning computing device 106. Computing device 200 includes one or more processors 201, working memory 202, one or more input/output devices 203, instruction memory 207, a transceiver 204, one or more communication ports 207, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Instruction memory 207 can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. Processors 201 can be configured to perform a certain function or operation by executing code, stored on instruction memory 207, embodying the function or operation. For example, processors 201 can be configured to execute code stored in instruction memory 207 to perform one or more of any function, method, or operation disclosed herein.

Additionally processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of radioablation diagnosis and treatment planning computing device 200. Working memory 202 can be a random access memory (RAM) such as a static random access memory (SRAM) or dynamic random access memory (DRAM), or any other suitable memory.

Input-output devices 203 can include any suitable device that allows for data input or output. For example, input-output devices 203 can include one or more of a keyboard, a touchpad, a mouse, a stylus, a touchscreen, a physical button, a speaker, a microphone, or any other suitable input or output device.

Communication port(s) 209 can include, for example, a serial port such as a universal asynchronous receiver/transmitter (UART) connection, a Universal Serial Bus (USB) connection, or any other suitable communication port or connection. In some examples, communication port(s) 209 allows for the programming of executable instructions in instruction memory 207. In some examples, communication port(s) 209 allow for the transfer (e.g., uploading or downloading) of data, such as image data.

Display 206 can be any suitable display, such as a 3D viewer or a monitor. Display 206 can display user interface 205. User interfaces 205 can enable user interaction with computing device 200. For example, user interface 205 can be a user interface for an application that allows a user (e.g., a medical professional) to view or manipulate models to define a target region of treatment for a patient as described herein. In some examples, the user can interact with user interface 205 by engaging input-output devices 203. In some examples, display 206 can be a touchscreen, where user interface 205 is displayed on the touchscreen. In some examples, display 206 displays images of scanned image data (e.g., image slices).

Transceiver 204 allows for communication with a network, such as the communication network 118 of FIG. 1. For example, if communication network 118 of FIG. 1 is a cellular network, transceiver 204 is configured to allow communications with the cellular network. In some examples, transceiver 204 is selected based on the type of communication network 118 radioablation diagnosis and treatment planning computing device 200 will be operating in. Processor(s) 201 is operable to receive data from, or send data to, a network, such as communication network 118 of FIG. 1, via transceiver 204

Referring back to FIG. 1, database 116 can be a remote storage device (e.g., including non-volatile memory), such as a cloud-based server, a disk (e.g., a hard disk), a memory device on another application server, a networked computer, or any other suitable remote storage. In some examples, database 116 can be a local storage device, such as a hard drive, a non-volatile memory, or a USB stick, to one or more of target definition computing device 104 and treatment planning computing device 106.

Communication network 118 can be a WiFi© network, a cellular network such as a 3GPP® network, a Bluetooth© network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. Communication network 118 can provide access to, for example, the Internet.

Imaging device 102 is operable to scan images, such as images of a patient's organs, and provide image data 103 (e.g., measurement data) identifying and characterizing the scanned images to communication network 118. Alternatively, imaging device 102 is operable to acquire electrical imaging such as cardiac ECG images. For example, imaging device 102 may scan a patient's structure (e.g., organ), and may transmit image data 103 identifying one or more slices of a 3D volume of the scanned structure over communication network 118 to one or more of target definition computing device 104 and treatment planning computing device 106. In some examples, imaging device 102 stores image data 103 in database 116, and one or more of target definition computing device 104 and treatment planning computing device 106 may retrieve the image data 103 from database 116.

In some examples, target definition computing device 104 is operable to communicate with treatment planning computing device 106 over communication network 118. In some examples, target definition computing device 104 and treatment planning computing device 106 communicate with each other via database 116 (e.g., by storing and retrieving data from database 116). In some examples, one or target definition computing devices 104 and one or more treatment planning computing devices 106 are part of a cloud-based network that allows for the sharing of resources and communication with each device.

In some examples, an electrophysiologist (EP) operates target definition computing device 104 to define a target region of treatment for a patient as described herein. In some examples, target definition computing device 104 generates target data identifying the target region for the patient, and transmits the target data to treatment planning computing device 106. A radiation oncologist may operate treatment planning computing device 106 to deliver treatment via imaging device 102 to the patient. In some examples, the target region is integrated into a radioablation treatment plan for treating the patient.

In some examples, one or target definition computing devices 104 are located in a first area 122 of a medical facility 120, while one or more target definition computing devices 104 are located in a second area 124 of the medical facility 120. As such, cardiac radioablation diagnosis and treatment system 100 allows multiple EPs to collaborate to finalize the target area. For example, one EP may operate a first target definition computing device 104 in a first medical facility 122, and a second EP may operate a second target definition computing device 102 in a second medical facility 124. First target definition computing device 104 and second target definition computing device 104 may communicate over communication network 118, such as by transmitting and receiving data related to (e.g., defining) the target area (e.g., a proposed target area). Each EP may operate the corresponding target definition computing device 104 to adjust the target area, and may finalize the target area once both EPs are in agreement of the target area.

### Study Generation

Target definition computing device 102 may execute an application that causes the generation of a user interface (e.g., user interface 205) which may be displayed to a medical professional, such as an EP. The executed application may allow the medical professional to define a target area of a patient for treatment. For example, the user interface allow the medical professional to select a study type (e.g., CT, ECG, MRI, etc.). The study type may identify a type of imaging for the patient. For example, the study type may identify a type of an image captured for the patient.

In response to the selection of the study type (e.g., via a drop down menu), the executed application automatically provides, via the user interface, a selection of a study category for the selected study type. The study category may identify a list of features (or study localizations) for a specific study type. For example, and assuming the medical profession selects “ECG” for the study type, the user interface may provide for the selection of one or more study categories, such as “Electrical.” As another example, a study category of “Structural” may be provided for “CT,” “MR,” “PET/SPECT,” and “US” study types. Additional study categories may include “Metabolic” or any other suitable study category. In some examples, only one study category may be available for a study type (e.g., such as “Electrical” for the “ECG” study type) and, as such, the executed application may automatically select the lone study category for the selected study type.

Once the study category is selected, the executed application may allow, via the user interface, for the selection of a study localization. The study localization may identify a general target area of a patient's organ to be treated, such as one or more segments of a heart. The study localizations displayed for selection may depend on the selected study category and/or study type. For example, and assuming a study type of “ECG” and a study category of “Electrical,” the executed application may provide, via the user interface, for a selection of one or more study localizations including “VT exit site,” “VT enter site,” and “VT enter and exit site,” among others. As another example, and assuming a study type of “CT” and a study category of “Structural,” the executed application may provide, via the user interface, for a selection of one or more study localizations including “Scar.”

In some examples, once the medical professional has selected a study type, a study category, and a study localization, the executed application may provide for display an interactive model of an organ or portion thereof, such as a 17 segment model representing a basal level, mid-cavity level, and cardiac apex of a heart's ventricle. The interactive model may allow the medical professional to select one or more portions of the organ to be treated. For example, and assuming the interactive model is the 17 segment model of a heart's ventricle, the interactive model may allow the medical professional to select one or more of the 17 segments (e.g., segments 1 through 17). The medical professional may select each segment by, for example, clicking (e.g., using an input/output device 203) on each segment. As each segment is selected, in some examples, the executed application may change a color of each segment, or provide some other indication that the segment has been selected. In some examples, the color of each selected segment is dependent on the selected study category. For example, the executed application may display in grey selected segments for a study category of “Structural,” and may display in orange selected segments for a study category of “Electrical.”

In some examples, the executed application may display a name of each portion of the organ. For example, the executed application may display the name of a segment of the 17 segment model as the medical professional drags a cursor over the segment.

In some examples, the user interface allows the medical professions to store a record in a database, such as database 116, where the record identifies the selected study type, study category, study localization, and any selected portions (e.g., segments) of the interactive model. In some examples, the executed application allows the medical professional to name the record, to select a study date, and further to provide notes associated with the record, all of which may be stored in the database as part of the record.

### Target Selection

The executed application may further allow the medical professional to identify a target area for treatment. For example, the executed application may display one or more study category maps, where each study category map (e.g., “heat map”) corresponds to a study category. Each study category map may identify one or more portions of a patient's organ, such as a 17 segment model of a heart's ventricle. Further, each study category map provides an indication of features (e.g., study localizations) previously identified for the patient that correspond to a study category. For example, an “Electrical map” may provide an indication of one or more arrhythmia origins identified on the “Electrical” type studies performed on the patient, while a “Structural map” may provide an indication of one or more scar positions identified in a “Structural” type studies performed on the patient. Data identifying previous studies for a patient may be stored in database 116, for example. Target definition computing device 104 may obtain the data to generate the study category maps.

In some examples, each study category map indicates a number of corresponding selections for each of one or more portions of the patient's organ. For example, and assuming a 17 segment model, the executed application may display each segment in a particular color based on a number of times that segment was selected as of clinical interest on that study category. For example, and for an “Electrical map,” segments that have never been selected (e.g., during a previous study) may be displayed in white, segments that have been selected up to a threshold amount (e.g., once) may be displayed in light orange, and segments that have been selected more than the threshold amount of times may be displayed in dark orange.

Each study category map may display the segments in varying colors (e.g., varying color shades) based on corresponding selection amount ranges. For example, and for a “Structural map,” segments that have never been selected may be displayed in white, segments that have been selected up to a threshold amount may be displayed in light grey, and segments that have been selected more than the threshold amount of may be displayed in dark grey. In some examples, the executed application further provides a bar graph indicating the ranges and corresponding colors to each study category map.

In some examples, the executed application may display each segment of a study category map in a particular color based on a percentage of times that segment was selected as a clinical interest. Target definition computing device 104 may obtain data for the patient from database 116, and may determine, for each study category (e.g., Electrical, Structural, etc.), a number of times each segment was selected across all study types. Based on the number of selections for each segment, target definition computing device 104 may determine a total number of selections for each study category. Further, for each segment, target definition computing device 104 may determine a percentage of times that segment was selected for the study category based on the number of studies for that particular study category and the total number of selections for that segment (e.g., (number of selections for segment/total number of studies)*100)).

For example, and for an “Electrical map,” segments with no previous selections may be displayed in white, segments with a percentage of “Electrical” study category selections up to a threshold amount may be displayed in light orange, and segments with a percentage of selections more than the threshold amount of “Electrical” study category selections may be displayed in dark orange. Similarly, and for a “Structural map,” segments with no previous selections may be displayed in white, segments with a percentage of “Structural” study category selections up to a threshold amount may be displayed in light grey, and segments with more than the threshold amount of “Structural” study category selections may be displayed in dark grey. In some examples, the executed application further provides a bar graph indicating the percentages and corresponding colors to each study category map.

The threshold amounts described herein may be configurable. For example, the medical professional may provide the threshold amounts to target definition computing device 104 via the user interface provided by the executed application, and target definition computing device 104 may store the thresholds in database 116.

In some examples, target definition computing device 104 generates a probability map which, in some examples, may be in the same form of a study category map. For example, if a study category map is a 17 segment model, the probability map may also be a 17 segment model. The probability map may indicate a probability of treatment for one or more portions of an organ based on those portions of the organ identified by one or more study category maps. In one example, target definition computing device 104 determines a number of selections provided to each portion (e.g., segment) of the organ regardless of study category (e.g., a total number of selections provided to a segment across all study categories). For example, the probability map may combine together two or more study category maps, and provide an indication of how many times one or more portions of an organ were selected (e.g., as indicated by the individual study category maps). Based on the determined number of selections for each portion, the executed application displays a corresponding portion of the probability map in a corresponding color or uses another suitable indication, such as a corresponding hatching.

In some examples, target definition computing device 104 determines a percentage of times that each portion was selected across all study categories. Based on the determined percentages for each portion, the executed application displays a corresponding portion of the probability map in a corresponding color, or uses any other suitable indication.

In some examples, target definition computing device 104 determines an average amount that each portion was selected across all study categories. For example, target definition computing device 104 may determine an average amount for each portion by determining a number of times the portion was selected across all study categories, and dividing by the number of study categories. Based on the determined averages for each portion, the executed application displays a corresponding portion of the probability map in a corresponding color, or uses any other suitable indication.

In some examples, target definition computing device 104 assigns a weight (e.g., multiplier) to each study category. For example, target definition computing device 104 may determine a number of selections for a first portion of the probability map as described above, and may multiply the total number of selections by a first value to determine a first weighted value. Similarly, target definition computing device 104 may determine a number of selections for a second portion of the probability map as described above, and may multiply the total number of selections by a second value to determine a second weighted value. The first value may be less than, or greater than, the second value. Based on the first weighted value and the second weighted value, target definition computing device 104 may display the corresponding portion of the probability map in a corresponding color, or uses any other suitable indication.

In some examples, target definition computing device 104 may weight each study category map equally, regardless of how many times a corresponding portion of the organ was selected in a corresponding study category. For example, target definition computing device 104 may display study category maps according to a percentage that each portion of an organ was selected within that study category as described above. Target definition computing device 104 may determine a value for each portion based on the percentages for that segment in each of the study categories. Based on the determined values for each portion, the executed application displays a corresponding portion of the probability map in a corresponding color, or uses any other suitable indication. In some examples, target definition computing device 104 weights the percentage (e.g., applies a multiplier) for each portion, and determines the values based on the weighted percentages. The multipliers may be different for at least two segments. In some examples, the executed application allows the medical professional to configure the multipliers. Target definition computing device 104 may store the multipliers in database 116.

In some examples, the executed application may generate a target definition model, which may be a 17 segment model of a heart's ventricle, to allow the medical professional to identify the target area for treatment (e.g., ablation areas). In some examples, the medical professional may select one or more portions of the target definition model to identify the target area. For example, and in the example of a 17 segment model, the medical professional may select a segment by clicking on the segment (e.g., using an input/output device 203). In some examples, the executed application changes a color of the selected segment, or may otherwise indicate the selected segment to the medical professional.

Further, in some examples, target definition computing device 104 may determine if a selected segment is “improbable” or unlikely to be selected based on the probability map and/or corresponding study category maps (e.g., the values used to generate the study category maps). For example, target definition computing device 104 may apply one or more rules (e.g., algorithms) to the values determined to generate the study category maps to determine if a selected portion is improbable. Data identifying and characterizing the rules may be stored in database 116, for example. As an example, one rule may specify that a selected portion (e.g., segment) that corresponds to a percentage in the probability map that is below a threshold is “improbable.” As another example, another rule may specify that a selected portion that corresponds to a number of selections as indicated in the probability map that is below a threshold is “improbable.” The rules are not confined to these examples, and any suitable rule may be employed.

In some examples, one or more trained machine learning models are applied to the patient's data to determine if a selected segment is improbable. For example, a machine learning model, such as a neural network or one based on decision trees, may be trained with historical patient data to determine probable areas of treatment. The trained machine learning model may be applied to a particular patient's historical data (e.g., treatment data stored in database 116) and the selected portion for the patient to classify the selected portion as probable or improbable. The models may be applied to a wide selection of diagnostic data such as medical images and electrical diagnostic studies (e.g., ECG, ECGI, old catheter maps, etc.).

For any selected segments determined to be “improbable,” the executed application generates a message (e.g., via a pop-up window) with a warning indicating the improbability of the selection. The medical professional may consider the warning, and may dismiss the warning upon providing an input via the user interface.

### Target Alignment

Based on the target definition model, target definition computing device 104 may generate a three dimensional (3D) model of the corresponding structure (e.g., organ). For example, assuming the target definition model is a two dimensional (2D) 17 segment model of a heart's ventricle, target definition computing device 104 may generate a 3D representation of the 17 segment model. The 3D model may identify a basal, mid-cavity, apical, and apex regions of the heart's ventricle. For example, the 3D representation of the 17 segment model may be based on the shape of a surface mesh of a left ventricle structure.

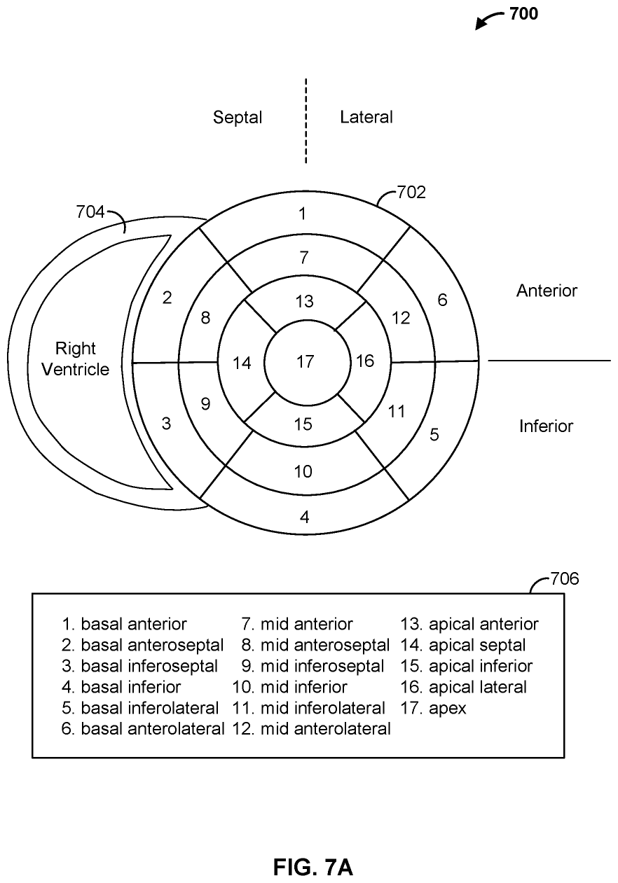

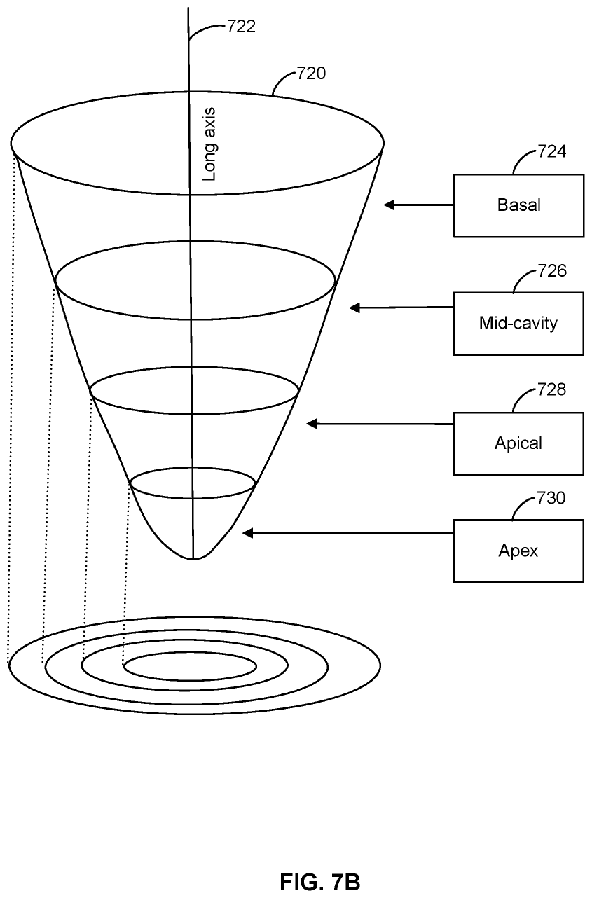

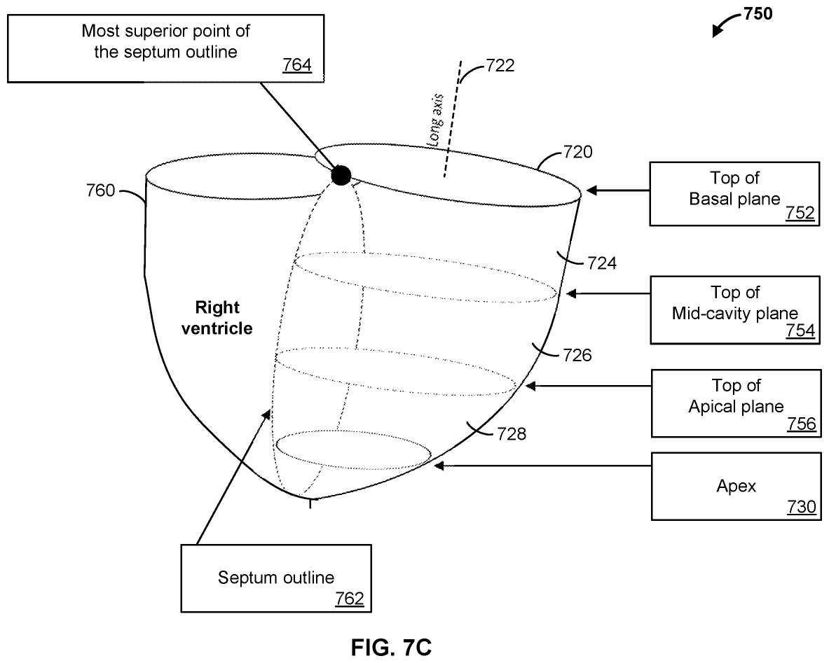

For example, FIG. 7A illustrates a 2D heart model 700 that includes a 2D ventricle model 702 adjacent a right ventricle model 704. As illustrated, 2D ventricle model 702 includes 17 segments, each segment identified by a corresponding number. A key 706 identifies the ventricle portions associated with each segment.

FIG. 7B illustrates a 3D ventricle model 720 that is a 3D representation of the 2D ventricle model 702. 3D ventricle model 720 identifies the basal 724, mid-cavity 726, apical 728, and apex 730 regions of the heart's ventricle, each portion including structure along a long axis 722 of the 3D model 720.

FIG. 7C illustrates a 3D heart model 750 that includes 3D ventricle model 720 adjacent a right ventricle model 760. The 3D ventricle model 720 includes a basal area 724 from the top of mid-cavity plane 754 to a top of basal plane 752, a mid-cavity area 726 from the top of apical plane 756 to top of mid-cavity plane 754, an apical area 728 from the top of apex 730 to top of apical plane 756. In addition, the 3D heart model 750 includes a septum border 762 defining an intersection between right ventricle model 760 and 3D ventricle model 720. Along the septum border 762 a most superior point 764 is illustrated where top of basal plane 752 contacts right ventricle 760.

Target definition computing device 104 may generate model data identifying and characterizing one or more of 2D model 702, 3D ventricle model 720, and 3D heart model 750, and store the data in database 116.

In some examples, a medical professional may provide input e.g., via input/output device 203) to target definition computing device 104 to adjust any one of 2D model 702, 3D ventricle model 720, and 3D heart model 750. The executed application may receive the input, and adjust a corresponding model as described herein.

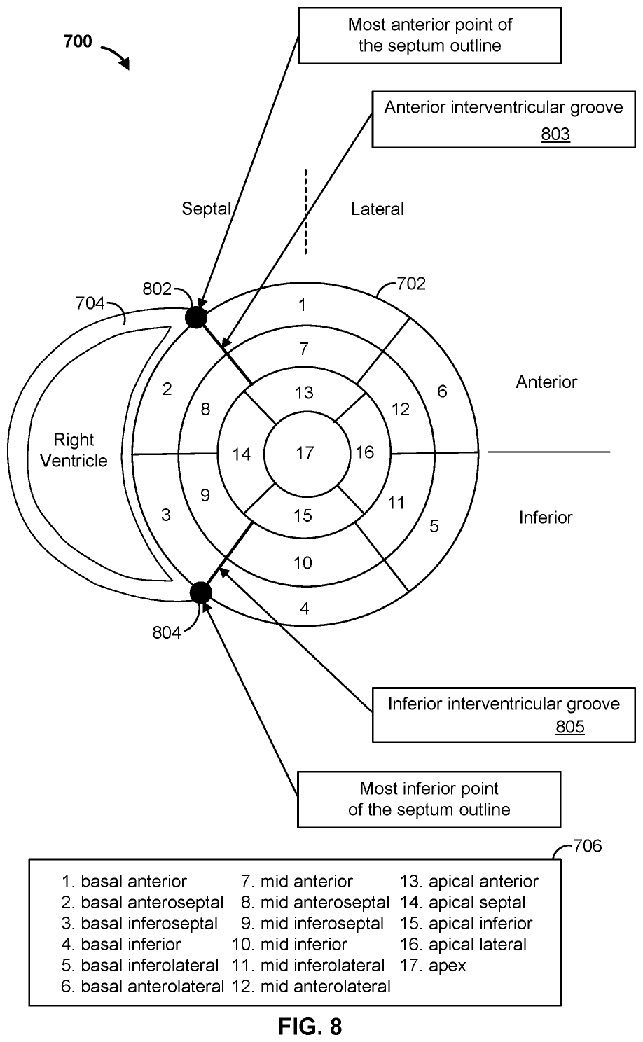

For example, FIG. 8 illustrates 2D heart model 700 with drag points 802, 804. A medical professional may provide input to target definition computing device 104 to adjust the location of anterior interventricular groove 803 by adjusting drag point 802. Similarly, the medical professional may provide input to target definition computing device 104 to adjust the location of inferior interventricular groove 805 by adjusting drag point 804. The drag points 802, 804 are configured to slide along an outer edge of 2D model 702.

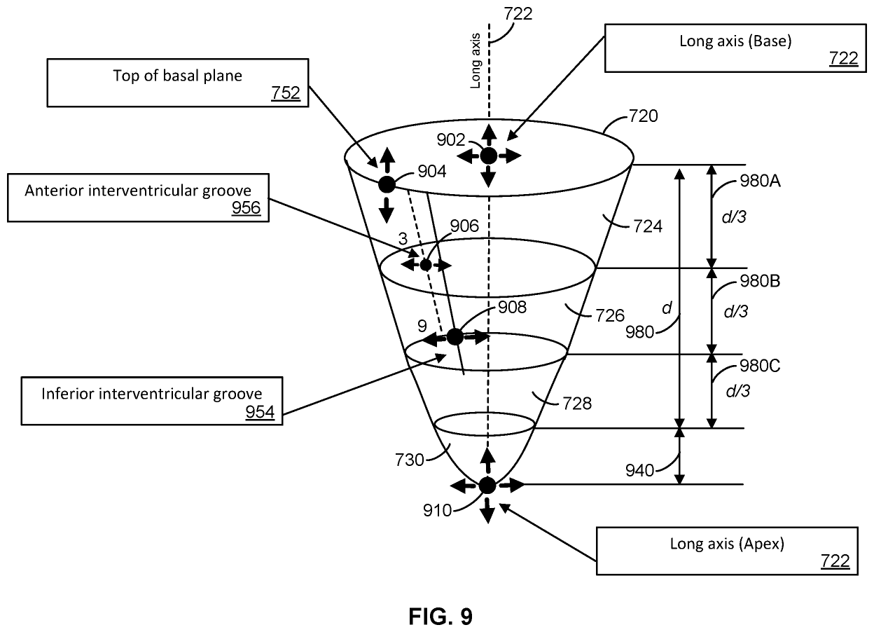

The medical professional may perform adjustments on 3D model, such as 3D ventricle 720. For example, FIG. 9 illustrates 3D ventricle model 720 with drag points 902, 904, 906, 908, 910 that allow for adjustment. The medical professional may adjust drag point 906 to adjust a location of the anterior interventricular groove 956. Similarly, the medical professional may adjust drag point 908 to adjust a location of the inferior interventricular groove 954. In this manner, an alignment with a ventricle, such as right ventricle 760, may be achieved.

The medical professional may also adjust an orientation of 3D ventricle model 720 by adjusting drag point 902. For example, if the medical professional drags drag point 902 to the right, 3D ventricle model 720 will “tilt” to the right (e.g., by a number of degrees). The medical professional may also adjust a length 980 by adjusting drag point 902 along long axis 722. For example, the medical professional may cause the elongation of 3D ventricle model 720 by dragging drag point 902 upwards, and may cause the shortening of 3D ventricle model 720 by dragging drag point 902 downwards. In some examples, an adjustment to length 980 causes an equal or near equal change in lengths 980A, 980B, 980C.

Dragging drag point 904 may cause the basal area 724 to elongate (e.g., by dragging drag point 904 upwards), or to shorten (e.g., by dragging drag point 904 downwards). For example, dragging drag point 904 may cause a change to length 980A. Likewise, dragging drag point 910 may cause the apex area 730 to elongate or shorten, causing a change to length 940.

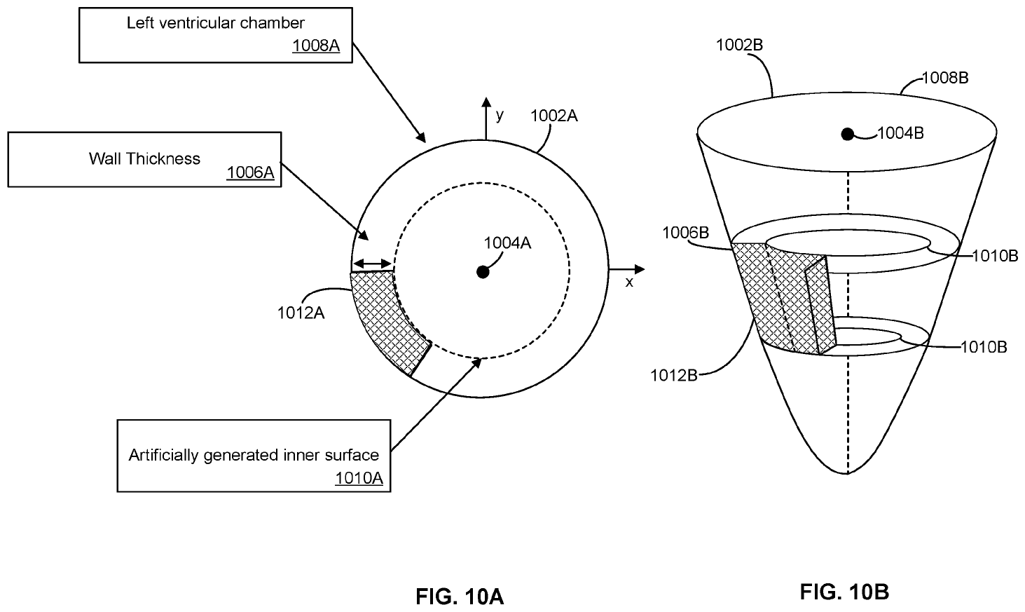

FIGS. 10A and 10B illustrate the generation of an ablation volume based on a selected target segment. For example, FIG. 10A illustrates a 2D segment model 1002A, which may be a target definition model. FIG. 10B illustrates a corresponding 3D segment model 1002B. 2D segment model 1002A illustrates a left ventricular chamber 1008 with a particular wall thickness 1006A (e.g., 10 millimeters) measured from an inner surface 1010A. Inner surface surrounds a center point 1004A. FIG. 10A further illustrates a selected segment 1012A (e.g., segment 9 of a 17 segment model of a heart ventricle), which the medical professional may have selected.

3D segment model 1002B includes left ventricular chamber 1008B with wall thickness 1006B measured from inner surface 1010B. Inner surface 1010B surrounds lateral line 1004B. Lateral line 1004B corresponds to center point 1004A. FIG. 10B also illustrates ablation volume 1012B, which corresponds to selected segment 1012A.

Thus, if a medical professional selects segment 1012A, target definition computing device 104 may automatically generate ablation volume 1012B for 3D segment model 1002B, and may display 3D segment model 1002B.

Referring back to FIG. 1, target definition computing device 104 may obtain image data 103 for the patient. The image data 103 includes an image of a scanned structure of the patient. For example, the image data 103 may include a 3D volume of a scanned structure of the patient. The scanned structure may correspond to the organ or portion thereof identified by the 3D representation model. Target definition computing device 104 may map the 3D model of the corresponding structure to the image of the scanned structure. For example, target definition computing device 104 may determine an initial alignment of the 3D model to the scanned structure of the image. To determine the initial alignment, target definition computing device 104 may execute an alignment algorithm. For example, the following describes an initial alignment of a 17-segment model with a left ventricle anatomy based on the following.

First, an interventricular septum outline on the left ventricle surface is identified by artificially expanding the uploaded left and right ventricles to detect the intersection of the surfaces. The long axis is determined based on the geometrical shape of the left ventricle and the orientation of the septum plane. The basal, mid-cavity, and apical section planes are then identified based on the following steps. The top of the basal plane is placed in correspondence of the most superior point of the septum outline, perpendicular to the long axis. The apex segment is placed at the extreme tip of the ventricle with a default thickness (e.g., 10 mm) along the long axis. The apical, mid-cavity, and basal planes are uniformly distributed along the long axis. Further, the segments are located based on the following steps. The position of the septal segments are determined by the anterior and posterior interventricular grooves, which are identified in correspondence of the most anterior and most inferior point of the interventricular septum outline. The other basal and mid-cavity segments are then uniformly distributed throughout the ventricular fee wall, in the basal and mid-cavity sections, respectively. Four segments of 90 degrees each are distributed in the apical section. They are placed such that the apical septal segment is centrally aligned with the basal and mid inferolateral and anterolateral segments.

Target definition computing device 104 may then superimpose the 3D model onto the image according to the determined alignment to generate a 3D structure image. The executed application may provide for display the 3D structure image (i.e., the image of the scanned structure superimposed with the 3D model).

Once mapped, the executed application allows the medical professional to adjust the alignment and/or the orientation of the 3D model to the image as described herein. For example, target definition computing device 104 may determine a long axis along the 3D model, and may further determine a border of a target region of treatment on the 3D model. The executed application may include one or more “drag points” along the 3D model, where the medical professional can drag (e.g., using input/output device 203) each point to a new location, thereby adjusting portions of the 3D model with respect to the structure in the image. The medical professional may also drag the long axis to a new position to alter an orientation of the 3D model with respect to the structure in the image.

In some examples, the 3D model includes a target region map that the medical professional can manipulate to define the target region of treatment for the patient (e.g., ablation areas). Initially, the target region map corresponds to image portions defined by the 3D model that correspond to the selected portions (e.g., segments) of the target definition model (e.g., a target region map). For example, if the medical professional selected segments 17 and 16 of a 17 segment model for ablation, target definition computing device 104 determines the corresponding segments as defined by the 3D model. In some examples, the executed application displays the target region map in a distinct color. Further, those portions of the scanned structure within the image that fall within the determined 3D portions may be displayed in a distinct color (e.g., red). The medical professional may adjust drag points to adjust the target region map. For example, the medical professional may adjust one or more drag points to define the contour of the target region map of the 3D model.

In some examples, target definition computing device 104 determines whether each medical professional adjustment violates one or more predetermined rules. If an adjustment violates a rule, the executed application may display a pop-up message with a warning. A rule may include, for example, determining whether the current alignment has strayed from the initial alignment by more than a threshold amount, such as by more than a threshold percentage. The medical professional may view and act on the warning, or may dismiss the warning. Application of the rules acts as a “sanity check” on each adjustment.

In some examples, the executed application allows the medical professional to select one or more other organs that may be displayed in conjunction with the 3D structure image. For example, the executed application may allow the medical professional to select for the display of an esophagus or lung adjacent to the 3D structure image of a heart's ventricle. The display of the other organs may include the display of 3D models of such organs. In some examples, the display includes scanned images of corresponding organs of the patient. These features may assist the medical professional during alignment, and may illustrate how other organs may be affected by proposed treatment (e.g., as identified by the ablation areas).

In some examples, the executed application allows for panning and zooming across the 3D structure image. In some examples, the executed application includes preconfigured selections (e.g., presets) for specific views of the 3D structure image). These preconfigured selections may be configurable by the medical professional.

Once the medical professional is complete with the alignment, the medical professional may provide an input to the executed application (e.g., via input/output device 203) to save the 3D structure image to a data repository, such as to database 116. In some examples, target definition computing device 104 transmit the 3D structure image to treatment planning computing device 106 to provide treatment to the patient based on the identified ablation areas.

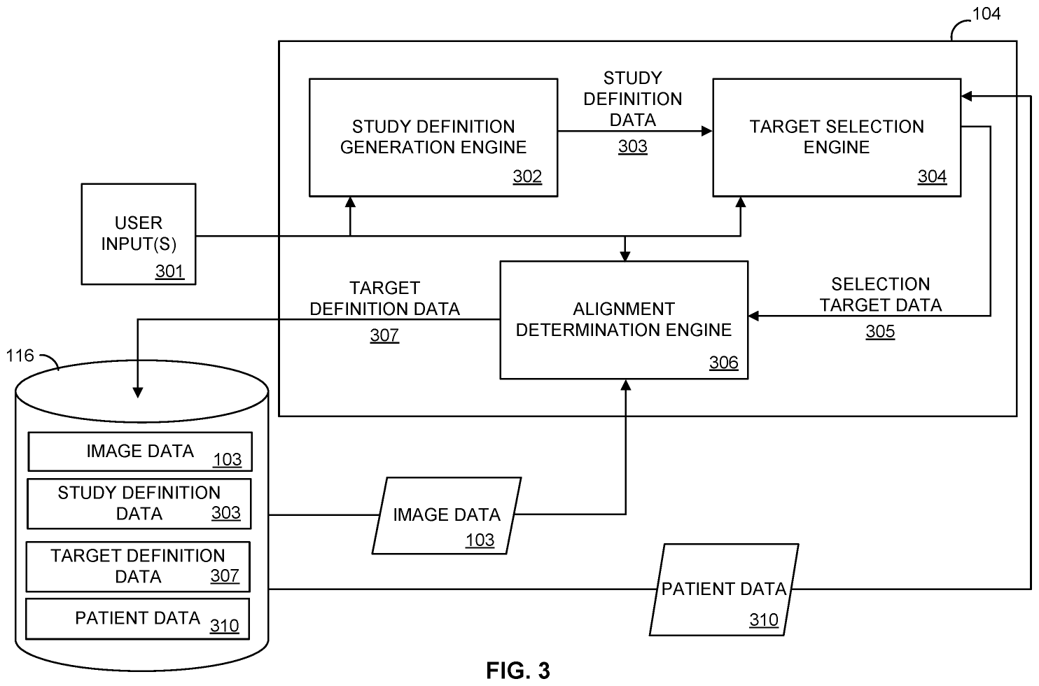

FIG. 3 illustrates exemplary portions of the cardiac radioablation diagnosis and treatment system of FIG. 1. In this example, target definition computing device 104 includes study definition generation engine 302, target selection engine 304, and alignment determination engine 306. In some examples, one or more of study definition generation engine 302, target selection engine 304, and alignment determination engine 306 may be implemented in hardware. In some examples, one or more of study definition generation engine 302, target selection engine 304, and alignment determination engine 306 may be as an executable program maintained in a tangible, non-transitory memory, such as instruction memory 207 of FIG. 2, that may be executed by one or processors, such as processor 201 of FIG. 2.

In this example, each of target definition computing device 104 includes study definition generation engine 302, target selection engine 304, and alignment determination engine 306 may receive user input(s) 301. For example, a medical professional may provide user input(s) 301 via input/output device 203, or via a touchscreen of display 206. User input(s) 301 may be received within a graphical user interface (GUI) provided by an executed application. Each of study definition generation engine 302, target selection engine 304, and alignment determination engine 306 may receive data from (e.g., user input(s) 301) the GUI, and may provide data to the GUI, such as data for display.

Study definition generation engine 302 may generate study definition data 303 identifying a study data record based on user input(s) 301. The study data record may identify a study type, a study category, a study localization, and any selected portions (e.g., segments) of an interactive model, as described herein. The study data record may also identify a name of the study data record, a date of the study data record, and any notes provided by a medical professional, as described herein. Study definition generation engine 302 provides the study definition data 303 to target selection engine 304. In some examples, study definition generation engine 302 stores the study definition data 303 in database 116.

Target selection engine 304 may perform operations to identify a target area for treatment. For example, target selection engine 304 may generate for display one or more study category maps, where each study category map (e.g., “heat map”) corresponds to a study category. Each study category map may identify one or more portions of a patient's organ, such as a 17 segment model of a heart's ventricle. In addition, target selection engine 304 may generate for display a probability map, which, in some examples, may be in the same form as a study category map. The probability map may indicate a probability of treatment for each portion of the organ (e.g., using different colors) based on those portions of the organ identified by the study category maps, as described herein. For example, target selection engine 304 may obtain patient data 310 from database 116 for a corresponding patient. The patient data 310 may identify previous studies the patient has received, as well as any study data record corresponding to that treatment. Based on patient data 310, target selection engine 304 may determine how probable a treatment for the patient is, as described herein.

Target selection engine 304 may further generate for display a target definition model, such as a 17 segment model of a heart's ventricle, to allow the medical professional to identify the target area for treatment (e.g., ablation areas). The medical professional may provide user input(s) 301 to select one or more portions of the target definition model to identify the target area. In some examples, target selection engine 304 determines if a selection is “improbable” as described herein, and provides for display (e.g., via a popup window) a warning regarding the selection when the selection is determined to be improbable. Target selection engine 304 generates selection target data 305 identifying the selected portions of the target definition model, and provides selection target data 305 to alignment determination engine 306.

Alignment determination engine 306 may perform operations to generate and provide for display a 3D model of the organ or portion thereof corresponding to the target definition model. Further, alignment determination engine 306 may obtain image data 103 for the patient identifying a corresponding scanned structure, such as a 3D image of the patient's heart ventricle. Alignment determination engine 306 may determine an alignment of the image to the 3D model, and may superimpose the 3D model onto the image according to the determined alignment to generate a 3D structure image. Alignment determination engine 306 may then provide the 3D structure image for display, such as for displaying on display 206.

Further, alignment determination engine 306 may receive user input(s) 301 identifying and characterizing adjustments to the 3D structure image. In response to the user input(s) 301, alignment determination engine 306 may adjust the 3D structure image accordingly. For example, alignment determination engine 306 may refine the alignment of the 3D model to the image, or may adjust drag points to define the target region map identifying the target area of treatment. Alignment determination engine 306 may generate target definition data 307 identifying and characterizing the 3D structure image, including the target region map, and may store target definition data 307 in database 116.

In some examples, alignment determination engine 306 determines whether each medical professional adjustment violates one or more predetermined rules. If an adjustment violates a rule, alignment determination engine 306 may cause the display of a pop-up message with a warning. In some examples, alignment determination engine 306 receives one or more user input(s) 301 identifying a selection of one or more other organs that may be displayed in conjunction with the 3D structure image. In response, alignment determination engine 306 provides for display 3D models of such organs. In some examples, alignment determination engine 306 provides for display image data 103 of the patient's corresponding organs.

In some examples, alignment determination engine 306 receives one or more user input(s) 301 identifying a pan or zoom action. In response, alignment determination engine 306 may pan or zoom across the 3D structure image. In some examples, alignment determination engine 306 receives one or more user input(s) 301 identifying the selection of a preconfigured selections for specific views of the 3D structure image. Alignment determination engine 306 may adjust the 3D structure image in accordance with the specific view selected, and may provide for display the adjusted 3D structure image.

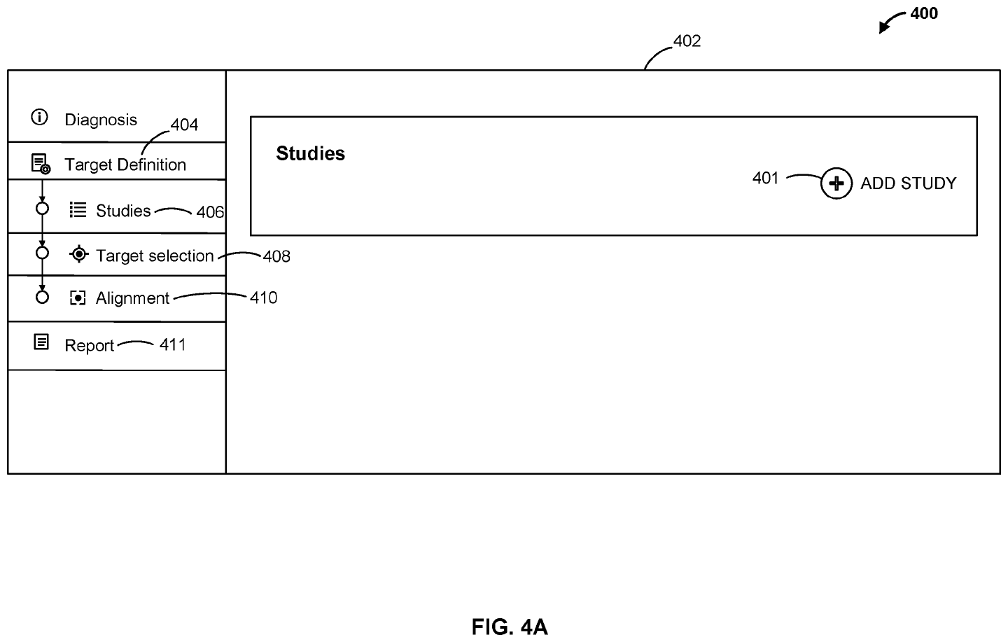

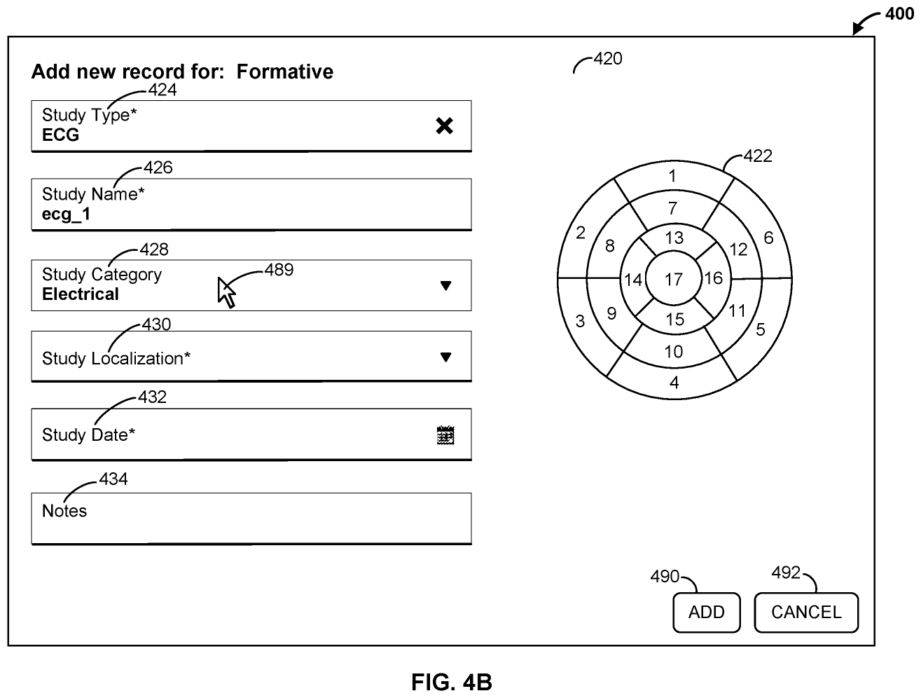

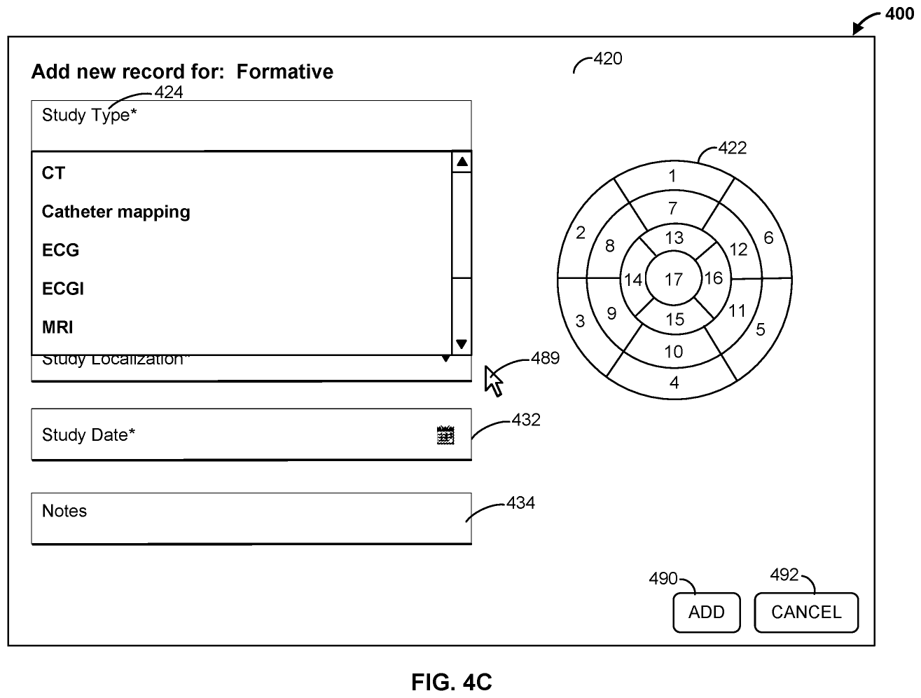

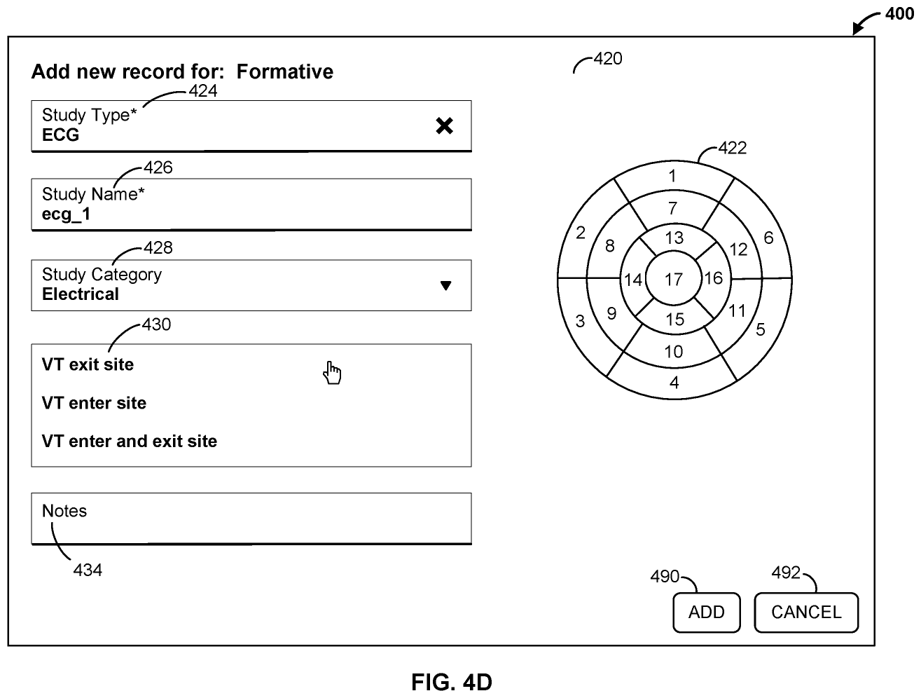

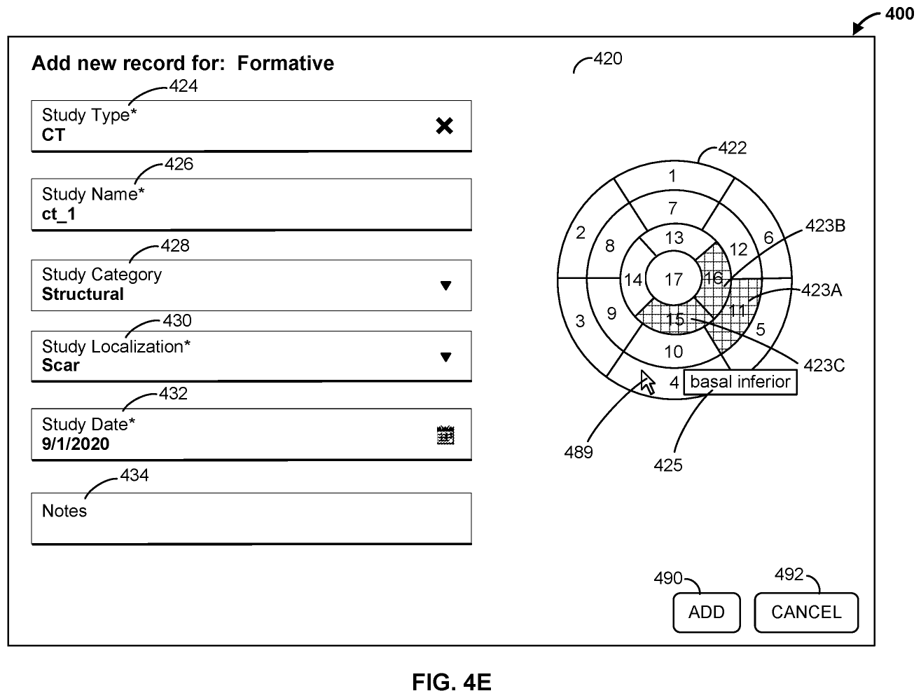

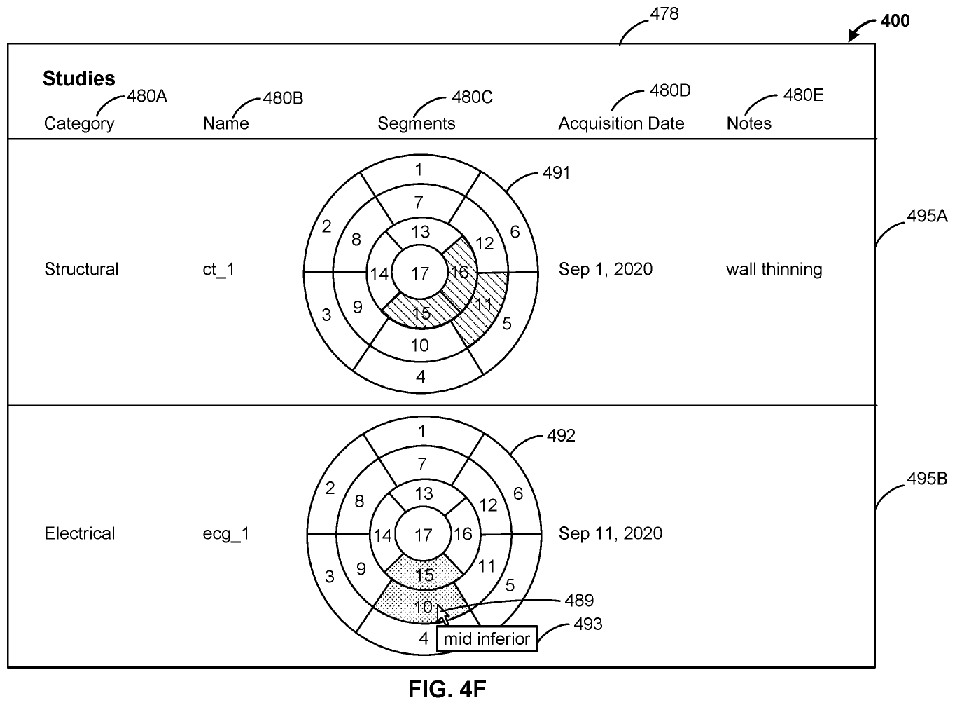

FIG. 4A illustrates a first portion 402 of a GUI 400 that allows a medical professional, such as an EP, to define a target area for treatment (e.g., ablation). The GUI 400 may be generated by an application executed by target definition computing device 104, and may be displayed to the medical professional on a display, such as display 206.

GUI 400 facilitates a number of steps to define a target area for treatment including generating a study data record, identify a target area for treatment, and align the target area to an image of a patient's organ. These steps are represented by studies icon 406, target selection icon 408, and alignment icon 410, each of which is illustrated under the target definition icon 404. Selecting one of studies icon 406, target selection icon 408, and alignment icon 410 may present to the user a portion of GUI 400 corresponding to that step.

To begin target definition, first portion 402 includes a study icon 401 that, if selected, allows for the generation of a new study data record. Page 402 also includes a report icon 411 that, if selected, generates a report based on the corresponding study data record. The report may include the study data record, any selected target areas (e.g., segments), a scanned image of the patient (e.g., scanned by image canning device 102), and data identifying and characterizing an alignment of the selected target area to the image of the patient's organ.

FIG. 4B illustrates a second portion 420 of GUI 400 that may be displayed when the medical professional selects the add study icon 401 of FIG. 4A. For example, the second portion 420 may be a pop-up window that is displayed upon the medical professional clicking on the add study icon 401. Second portion 420 incudes a study type drop-down menu 424, a study category drop down menu 428, and a study localization drop down menu 430.

Study type drop-down menu 424 allows the medical professional to select a study type for the study type record. For example, and as illustrated in FIG. 4B, study type drop-down menu 424 may allow the medical professional to select from a plurality of study types (e.g., imaging types), such as CT, Catheter Mapping, ECG, ECGI, and MRI, among others.

Once the medical professional selects a study type, GUI 400 automatically determines one or more study categories based on the selected study type. Each study category may identify a list of features (or study localizations) for a specific study type. The medical professional may view the available study categories available using study category drop-down menu 426. For example, and as illustrated in FIG. 4D, the medical professional may select a study category of “Electrical” when the study type is “ECG.”

Once the study category is selected, GUI 400 automatically determines one or more study localizations based on the selected study categories and/or selected study type. The study localization may identify a general target area of a patient's organ to be treated, such as one or more segments of a heart. For example, and as illustrated in FIG. 4D, study localization drop down menu 430, the medical professional may select a study localization of “VT exit site,” “VT enter site,” and “VT enter and exit site” when the selected study type is “ECG” and the selected study category is “Electrical.”

Referring back to FIGS. 4B, 4C, and 4D, second portion 420 also includes a study name text box 426 that allows the medical professional to provide a name for the study record, a study date selection box 432 that allows for the selection of a date (e.g., a current date), and a notes text box 434 that allows the medical professional to enter in notes (e.g., treatment notes, reminders, notes to other medical professionals, etc.).

In addition, second portion 420 includes an interactive model 422, which in this example is a 17 segment model representing segments of a heart's ventricle. The medical professional may select one or more portions of the interactive model 422, which may be areas for treatment. For example, and as illustrated in FIG. 4E, the medical professional may select a first segment 423A (e.g., segment 11), second segment 423B (e.g., segment 16), and a third segment 423C (e.g., segment 15). In addition, in some examples, when the cursor 489 is placed over a segment (e.g., segment 4), GUI 400 displays the name of the segment (e.g., via a pop-up window). In this example, cursor 489 appears over segment 4 of interactive model 422, and in response GUI 400 displays name box 425 identifying segment 4 as the “basal inferior” portion of a heart's ventricle.

To create the study data record, the medical professional may click on add icon 490. In response, 104 generates data identifying and characterizing the information provided to GUI 400, and stores the generated data in a data repository, such as within database 116. If the medical professional would like to start over and not save the study data record, the medical professional may click on the cancel icon 492, which results in the clearance of any provided inputs, and, in some examples, the display of first portion 402 as illustrated in FIG. 4A.

Referring to FIG. 4F, GUI 400 may include a third portion 478 that displays summaries of generated study data records. For example, GUI 400 may display portion 478 in response to the medical professional clicking on the add icon 490 of FIG. 4E. In some examples, GUI 400 displays portion 478 in response to the medical professional clicking the studies icon 406 of FIG. 4A.

Third portion 478 includes study category 480A, study name 480B, selected segments 480C, acquisition date 480D, and notes 480E display areas for each study data record generated. The study category 480A corresponds to the selected study category 428 for each study data record generated. Similarly, the study name 480B, acquisition date 480D, and notes 480E correspond to the study name 426, study date 432, and notes 434 for each study data record.

In this example, two summaries are illustrated including a first study summary 495A and a second study summary 495B. First study summary 495A includes a study category 480A of “Structural,” as well as corresponding interactive model 491 illustrating selected segments 11, 15, and 16. Second study summary 495B includes a study category 480A of “Electrical,” as well as corresponding interactive model 4912 illustrating selected segments 10 and 15. In some examples, when cursor 489 is placed over a corresponding portion of an interactive model, GUI 400 displays the name of the segment (e.g., via a pop-up window). In this example, cursor 489 appears over segment 10 of interactive model 492, and in response GUI 400 displays a name box 493 identifying segment 0 as the “mid inferior” portion of a heart's ventricle.

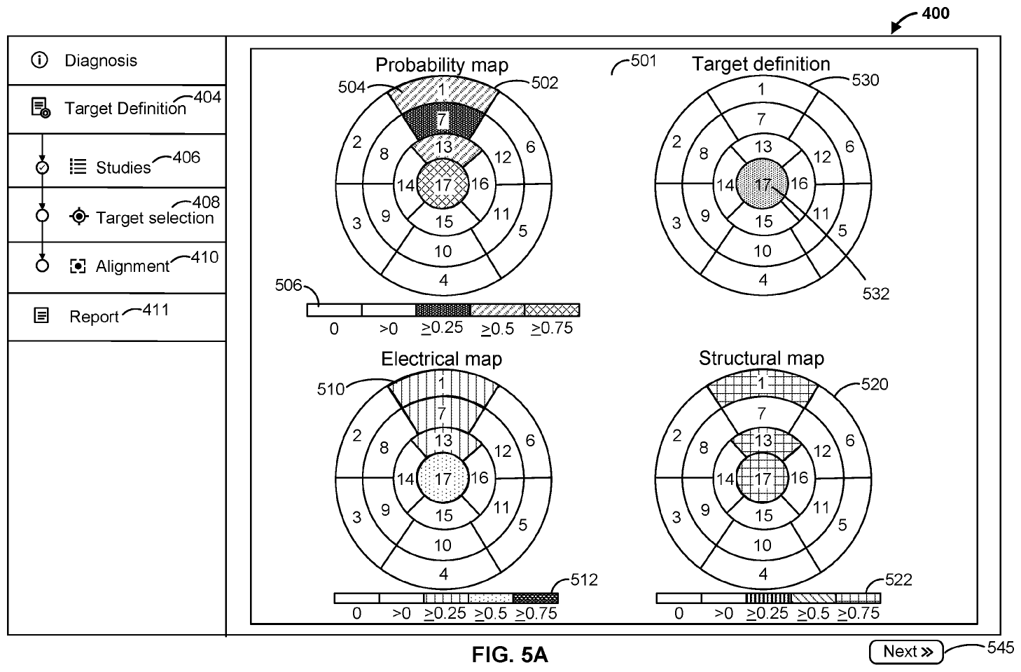

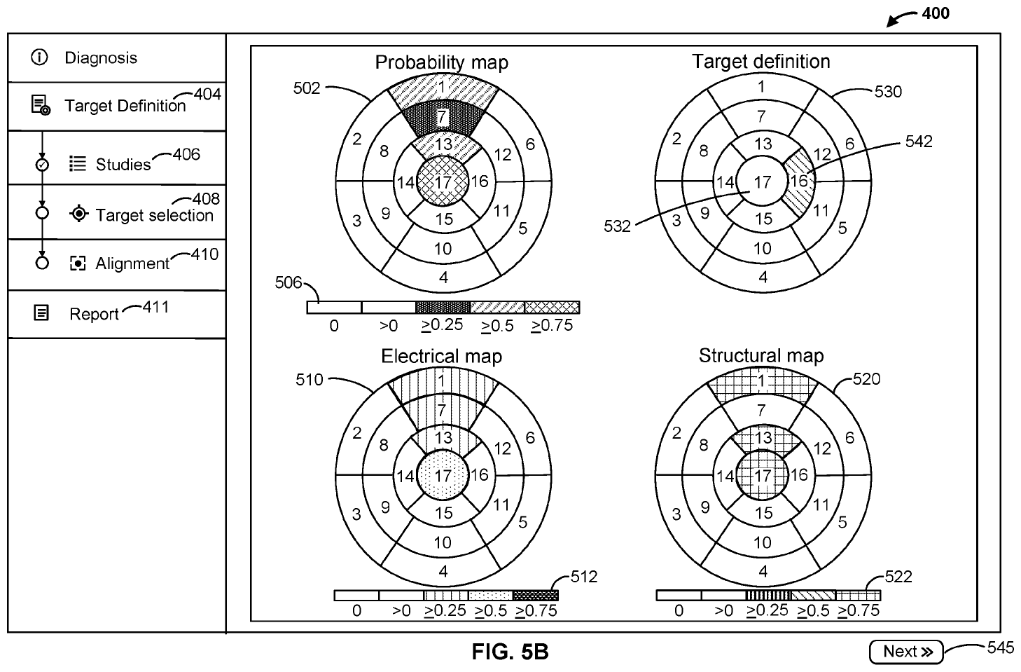

FIG. 5A illustrates a target selection portion 501 of GUI 400. Once a study data record is generated, for example, as discussed above with respect to FIGS. 4A-4F, GUI 400 may display target selection portion 501 to the medical professional. In some examples, GUI 400 displays target selection portion 501 in response to the medical professional clicking the target selection icon 408 of FIG. 4A.

In this example, target selection portion 501 displays a first study category map 510, which is based on a study category 428 of “Electrical,” and a second study category map 520, which is based on a study category 428 of “Structural.” As described herein, each study category map 510, 520 may identify one or more portions of a patient's organ, such as a 17 segment model of a heart's ventricle. Further, each study category map 510, 520 provides an indication of previous studies performed on the patient corresponding to the corresponding study category. In addition, each study category map 510, 520 is displayed with a corresponding bar graph 512, 522, respectively. Each bar graph 512, 522 indicates treatment amount ranges determined for each study category as described herein, and their corresponding hatching used within the segments of each respective study category map 510, 520.

Target selection portion 502 also includes a probability map 502 that indicates a probability of treatment for one or more portions of the patient's organ (in this example, the patient's heart) based on those portions of the organ identified by study category maps 510, 520. Probability map 502 is displayed with a corresponding bar graph 506 that indicates treatment segment probability ranges as described herein, and their corresponding hatching used within segments of the probability map 502.

Further, target selection portion 502 includes a target definition map 530 that, in this example, is in the form of a 17 segment model of a heart's ventricle. Target definition map 530 allows the medical professional to identify a target area for treatment. For example, the medical professional may select (e.g., using input/output device 203 to manipulate cursor 489) a segment of target definition map 530 to identify the target area 532. In this example, target area 532 includes segment 17 of the target definition map 530.

FIG. 5B is similar to FIG. 5A, but the medical professional may selects segment 16 of target definition map 530 to identify target area 542. Once the medical professional has identified the target area 532, 542 by selecting portions of target definition map 530, the medical professional may proceed to the next step be clicking on next icon 545.

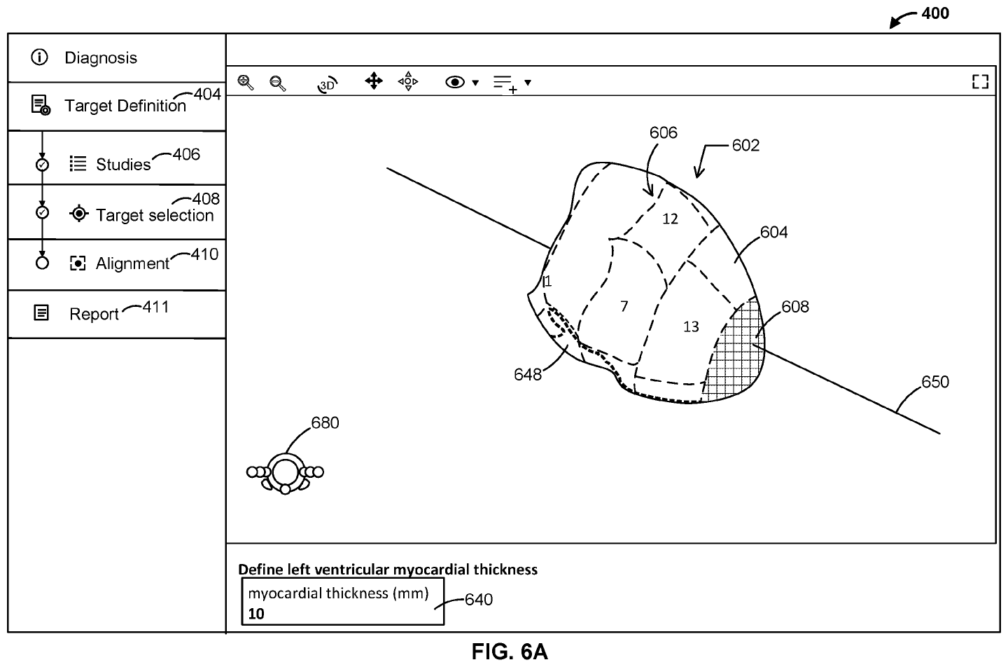

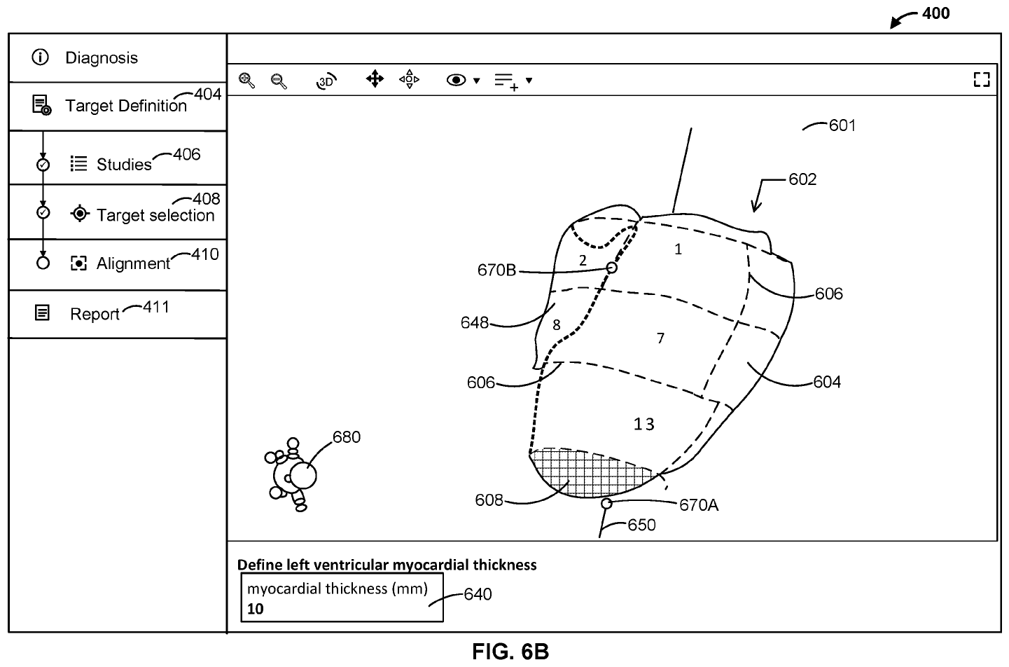

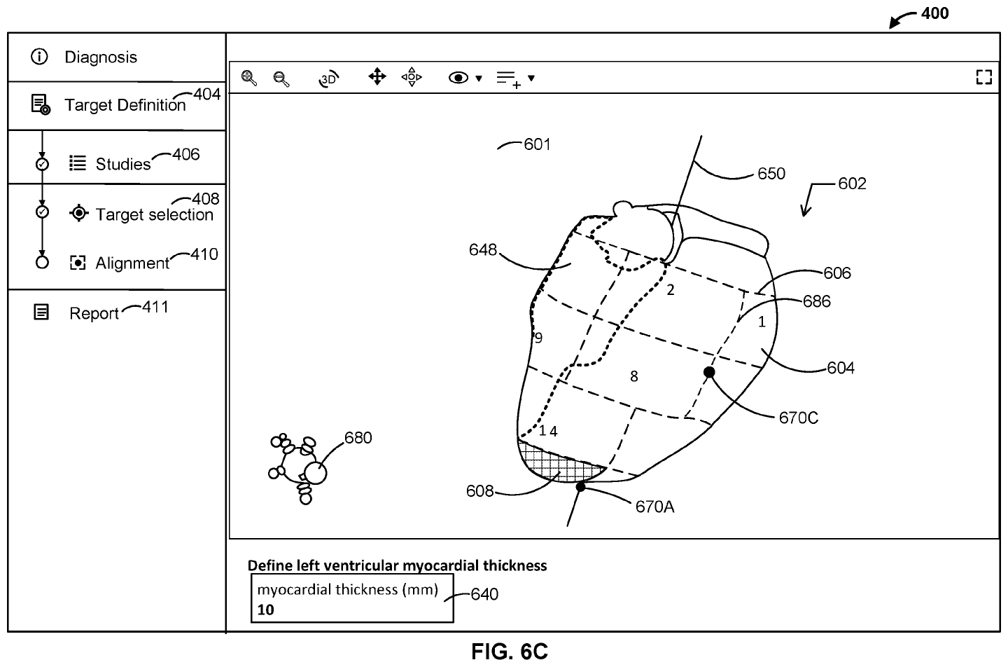

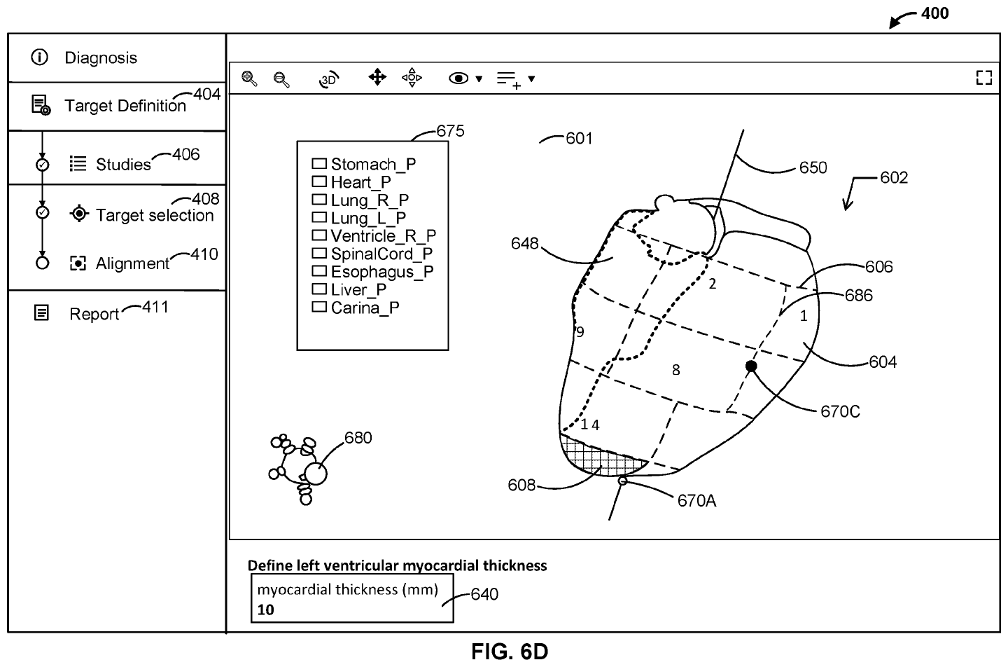

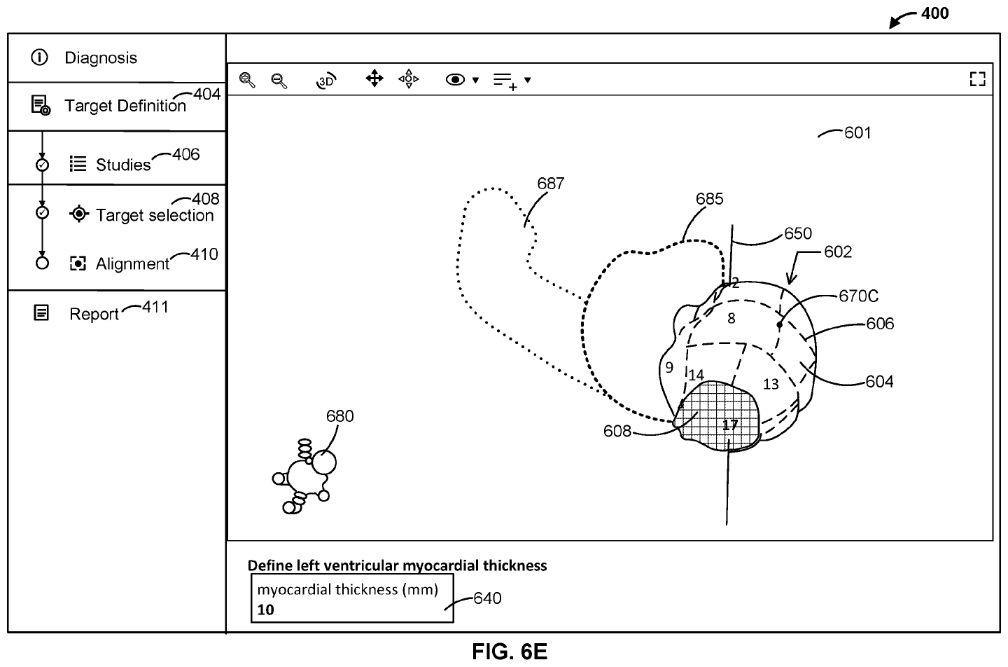

FIG. 6A illustrates an alignment portion 601 of GUI 400 that displays a 3D structure image 602 that includes a 3D segment model 606 superimposed onto scanned image 604. 3D segment model 606 may be a 3D segment model of a heart's ventricle, for example. Scanned image 604 may be an image scanned by image scanning device 102, such as a 3D volume of a scanned structure of the patient. 3D structure image 602 also includes a target region map 648, which defines a target region for treatment for the patient. The target region map 648 may correspond to one or more selected target areas of a target definition map, such as target areas 532, 542 of target definition map 530, at least initially (e.g., before adjustment by the EP). In some examples, target region map 648 is displayed in a distinct color. In some examples, a distinct hatching is used to display target region map 648, or any other suitable mechanism that allows the EP to easily determine the contours of target region map 648. Further, as displayed, a longitudinal axis 650 proceeds through an apex 608 of 3D structure image 602.

Alignment portion 601 may, in some examples, also display a reference character 680. The reference character 680 is displayed from a view according to an orientation of 3D structure image 602. For example, if the orientation of 3D structure image 602 is such that it is being displayed from an overhead view as the corresponding organ is positioned in the patient, then reference character 680 is displayed from an overhead view. This allows the EP to easily determine from what view and/or orientation 3D structure image 602 is currently being displayed.

Alignment portion 601 may, in some examples, include a text entry box 640 that allows for the entry of a value. In this example, the value entered is a myocardial thickness (e.g., left ventricular myocardial thickness). The myocardial thickness may be used to reconstruct the inner ventricular myocardium surface, where all identified target segments are projected, as described here. For example, target definition computing device 104 may execute an algorithm to generate a final 3D target volume by combining all regions bounded by selected segments and underlying projections. If a user (e.g., an EP) has not edited the myocardinal thickness, a default value, such as 10 mm, is used. For example, target definition computing device 104 may generate the 3D target volume based on selected segments as described herein. For example, in the example of a heart, target definition computing device 104 may take selected segments (e.g., which may be part of the epicaridial wall) and extrude a volume toward the center of the left ventricle with a depth based on a wall thickness definition.

In some examples, alignment portion 601 includes one or more adjustment icons 655 that allow for an adjustment of 3D structure image 602. For example, adjustment icons 655 may allow for zoom in, zoom out, panning, and rotating functionalities.

With reference to FIG. 6B, alignment portion 601 may display one or more drag points, such as drag points 670A, 670B, that allow the EP to make adjustments to 3D structure image 602. For example, the EP may adjust longitudinal axis 650 by dragging drag point 670A to a new location. In response, GUI 400 adjust an orientation of scanned image 604 with respect to 3D segment model 606. Similarly, the EP may adjust target region map 648 my dragging drag point 670B to a new location.

In some examples, GUI 400 allows for the creation, or removal, of drag points. For example, the EP may right-click on a drag point, such as drag point 670B, and select a “remove” option to remove the drag point. Likewise, the EP may right-click on a portion of 3D segment model 606, and select an “ad” option to add a drag point.

FIG. 6C illustrates 3D structure image 602 after the EP provided input to rotate 3D structure image 602 clockwise around longitudinal axis 650. In this example, drag point 670C may allow the EP to adjust an anterior interventricular groove 686 of 3D structure image 602.

Adjustment icons 655 may also allow the EP to display images of additional organs, such as organs that are adjacent to the organ identified by scanned image 604. For example, and with reference to FIG. 6D, the EP may select an adjustment icon 655 to display organ selection box 675, which allows the EP to select from one or more organs to display.

For example, and assuming the EP selects “lung” (e.g., “lung_r_p” for right lung, or “lung_l_p” for left lung) and “esophagus,” GUI 400 may display renderings (e.g., 3D renderings) of a first organ 685 (e.g., lung) and a second organ 687 (e.g., esophagus), as illustrated in FIG. 6E. The renderings may be 3D models pre-stored in database 116, for example. In other examples, the renderings are scanned images of the corresponding structure of the patient.

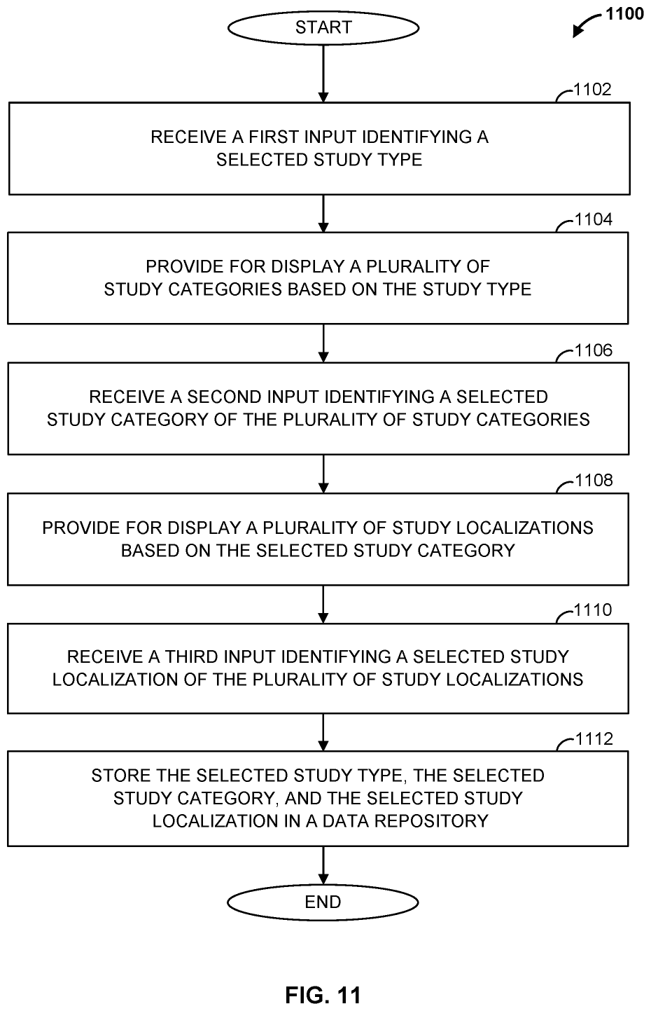

FIG. 11 is a flowchart of an example method 1100 that can be carried out by, for example, target definition computing device 104. Beginning at step 1102, a first input is received. The first input identifies a selected study type. For example, an EP may use input/output device 203 to provide an input to an executed application displaying a GUI, such as GUI 400, on display 206. The EP may select a study type 424 displayed within a portion 420 of GUI 400.

At step 1104, a plurality of study categories are provided for display. The plurality of categories are determined based on the selected study type. For example, GUI 400 may display the plurality of study categories within a study category dropdown menu 428. Proceeding to step 1106, a second input is received. The second input identifies a selected study category of the plurality of study categories. For example, the EP may select one of the plurality of study categories displayed within study category dropdown menu 428.

At step 1108, a plurality of study localizations are provided for display. The plurality of study localizations are determined based on the selected study category. For example, GUI 400 may display the plurality of study localizations within a study localization dropdown menu 430. At step 1110, a third input is received. The third input identifies a selected study localization of the plurality of study localizations. For example, the EP may select one of the plurality of study localizations displayed within study localization dropdown menu 430.

Proceeding to step 1112, the selected study type, the selected study category, and the selected study localization are stored in a data repository. For example, 104 may generate a study data record identifying the selected study category, and the selected study localization, and may store the study data record in database 116. The method then ends.

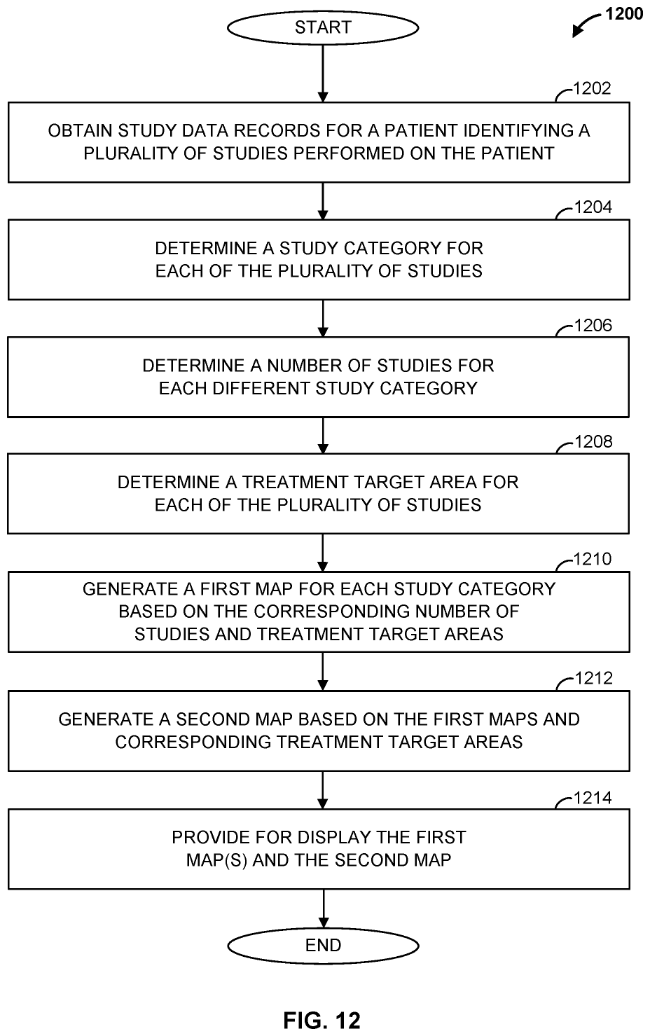

FIG. 12 is a flowchart of an example method 1200 that can be carried out by, for example, target definition computing device 104. Beginning at step 1202, study data records for a patient are obtained. The study data records identify a plurality of studies performed on the patient. For example, target definition computing device 104 may obtain study definition data 303 from database 116 for the patient. At step 1204, a study category is determined for each of the plurality of studies. For example, each of the plurality of studies may be associated with a study category, such as “Electrical” or “Structural.” At step 1206, a number of studies for each different category is determined. Further, at step 1208, a treatment target area for each of the plurality of studies is determined. For example, each of the plurality of studies may be associated with one or more segments targeted for treatment.

Proceeding to step 1210, a first map is generated for each study category based on the corresponding number of studies and treatment target areas. For example, target definition computing device 104 may determine, for each study category, a percentage of the corresponding number of studies treating each of a plurality of segments of the patient's organ. Each of the first maps may be, for example, study category maps 510, 520.

At step 1212, a second map is generated. The second map is generated based on the first maps and corresponding treatment target areas. For example, the second map may indicate probabilities of studies for one or more portions of the patient's organ based on those portions identified in the first maps. The second map may be, for example, probability map 502 that indicates a probability of treatment for one or more portions of the patient's organ based on those portions of the organ identified by study category maps 510, 520.

At step 1214, the first maps and the second map are provided for display. For example, the first maps and second map may be displayed within a target selection portion 501 of GUI 400. The method then ends.

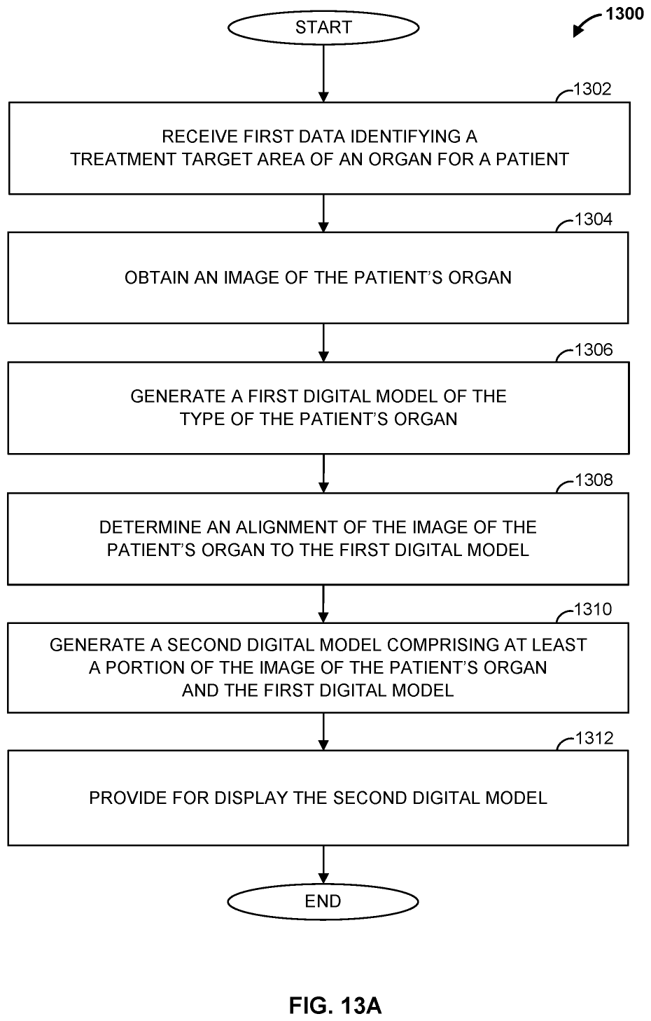

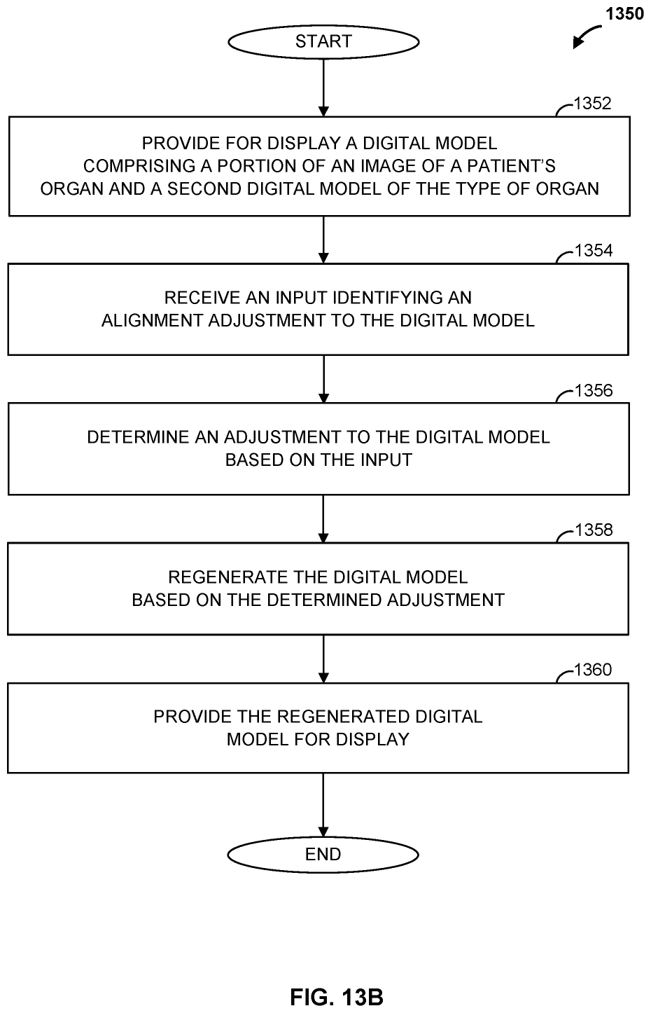

FIG. 13A is a flowchart of an example method 1300 that can be carried out by, for example, target definition computing device 104. At step 1302, first data is received. The first data identifies a treatment target area of an organ for a patient. For example, target definition computing device 104 may determine the treatment target area based on a segment of a target definition map 530 that an EP has selected to identify target area 532. At step 1304, an image of the patient's organ is obtained. For example, target definition computing device 104 may obtain an image, such as an image of a 3D volume, of an organ of the patient scanned by image scanning device 102.

Proceeding to step 1306, a first digital model of the type of the patient's organ is generated. For example, target definition computing device 104 may generate a 3D model, such as 3D ventricle model 720 or 3D segment model 1002B, of the patient's organ. At step 1308, an alignment of the image of the patient's organ to the first digital model is determined. Further, and at step 1310, a second digital model is generated. The second digital model comprises at least a portion of the image of the patient's organ and the first digital model. For example, target definition computing device 104 may superimpose 3D segment model 606 onto scanned image 604 to generate 3D structure image 602. At step 1312, the second digital model is provided for display. For example, target definition computing device 104 may display the second digital model to the EP. The method then ends.

FIG. 13B is a flowchart of an example method 1350 that can be carried out by, for example, target definition computing device 104. Beginning at step 1352, a digital model is provided for display. The digital model comprises a portion of an image of a patient's organ and a second digital model of the type of the organ. For example, the digital model may be generated in accordance with method 1300 of FIG. 13A. At step 1354, an input is received. The input identifies an alignment adjustment to the digital model. For example, 104 may receive an input from an EP using input/output device 203 to make an adjustment to 3D structure image 602 by dragging one or more drag points 670 as described herein.

Proceeding to step 1356, an adjustment to the digital model is determined based on the input. For example, the adjustment may be a change to an orientation of the image of the patient's organ with respect to the second digital model. The EP may adjust the orientation by dragging one or more drag points 670 to move a longitudinal axis 650, for example. In some examples, the adjustment may be a change to a target region map of the digital model. For example, the adjustment may be to target region map 648 of 3D structure image 602.

At step 1358, the digital model sis regenerated based on the determined adjustment. Further, and at step 1360, the regenerated digital model is provided for display. In some examples, 104 transmits the regenerated digital model to radioablation treatment system 126 for treating the patient. The method then ends.

In some examples a system includes a computing device. The computing device is configured to receive a first input identifying an organ of a patient, and receive a scanned image of the organ. The computing device is also configured to generate a first digital model of a type of the organ. Further, the computing device is configured to determine an alignment of the scanned image to the first digital model. The computing device is also configured to generate a second digital model comprising at least a portion of the scanned image and the first digital model. The computing device is further configured to store the second digital model in a data repository. In some examples, receiving the first input is in response to a selection of a portion of a displayed target definition map. In some examples, the organ is a heart. In some examples, the computing device is configured to provide the second digital model for display.

In some examples, the computing device is configured to receive a second input identifying an adjustment to the alignment of the scanned image to the first digital model. The computing device is also configured to adjust the second digital model based on the second input. The computing device is further configured to store the adjusted second digital model in the data repository.

In some examples, the computing device is configured to receive a second input identifying a treatment target area of the organ. The computing device is also configured to determine a corresponding portion of the second digital model based on the treatment target area of the organ. Further, the computing device is configured to regenerate the second digital model to identify the corresponding portion. In some examples, regenerating the second digital model includes associating the corresponding portion with a distinctive feature for display. In some examples, the computing device is further configured to transmit treatment data identifying the treatment target area of the organ to a radioablation treatment system.

In some examples, the computing device is configured to obtain study data records for the patient, wherein each study data record identifies one of a plurality of study types and a study target area of a plurality of study target areas for studies performed on the patient. The computing device is also configured to determine a first number of each of the plurality of study types performed on the patient based on the study data records. Further, the computing device is configured to determine, for each of the plurality of study types, a second number of studies performed on the patient in each of the plurality of study target areas. The computing device is also configured to generate a first map for each of the plurality of study types based on the corresponding first number and second numbers. The computing device is further configured to store the first map in the data repository. In some examples, each first map indicates a frequency of the corresponding study type on each of the plurality of study target areas. In some examples, the computing device is further configured to generate a second map based on the first numbers and the second numbers, wherein the second map indicates a probability of treatment for each of the plurality of study target areas, and to store the second map in a data repository.

In some examples, a computer-implemented method includes receiving a first input identifying an organ of a patient, and receive a scanned image of the organ. The method also includes generating a first digital model of a type of the organ. Further, the method includes determining an alignment of the scanned image to the first digital model. The method also includes generating a second digital model comprising at least a portion of the scanned image and the first digital model. The method further includes storing the second digital model in a data repository. In some examples, receiving the first input is in response to a selection of a portion of a displayed target definition map. In some examples, the organ is a heart. In some examples, the method includes providing the second digital model for display.

In some examples, the method includes receiving a second input identifying an adjustment to the alignment of the scanned image to the first digital model. The method also includes adjusting the second digital model based on the second input. The method further includes storing the adjusted second digital model in the data repository.

In some examples, the method includes receiving a second input identifying a treatment target area of the organ. The method also includes determining a corresponding portion of the second digital model based on the treatment target area of the organ. Further, the method includes regenerating the second digital model to identify the corresponding portion. In some examples, regenerating the second digital model includes associating the corresponding portion with a distinctive feature for display. In some examples, the method includes transmitting treatment data identifying the treatment target area of the organ to a radioablation treatment system.

In some examples, the method includes obtaining study data records for the patient, wherein each study data record identifies one of a plurality of study types and a study target area of a plurality of study target areas for studies performed on the patient. The method also includes determining a first number of each of the plurality of study types performed on the patient based on the study data records. Further, the method includes determining, for each of the plurality of study types, a second number of studies performed on the patient in each of the plurality of study target areas. The method further includes generating a first map for each of the plurality of study types based on the corresponding first number and second numbers. The method also includes storing the first map in the data repository. In some examples, each first map indicates a frequency of the corresponding study type on each of the plurality of study target areas. In some examples, the method includes generating a second map based on the first numbers and the second numbers, wherein the second map indicates a probability of treatment for each of the plurality of study target areas, and storing the second map in a data repository.

In some examples, a non-transitory computer readable medium stores instructions that, when executed by at least one processor, cause the at least one processor to perform operations including receiving a first input identifying an organ of a patient, and receive a scanned image of the organ. The operations also include generating a first digital model of a type of the organ. Further, the operations include determining an alignment of the scanned image to the first digital model. The operations also include generating a second digital model comprising at least a portion of the scanned image and the first digital model. The operations further include storing the second digital model in a data repository. In some examples, receiving the first input is in response to a selection of a portion of a displayed target definition map. In some examples, the organ is a heart. In some examples, the operations include providing the second digital model for display.

In some examples, the operations include receiving a second input identifying an adjustment to the alignment of the scanned image to the first digital model. The operations also include adjusting the second digital model based on the second input. The operations further include storing the adjusted second digital model in the data repository.

In some examples, the operations include receiving a second input identifying a treatment target area of the organ. The operations also include determining a corresponding portion of the second digital model based on the treatment target area of the organ. Further, the operations include regenerating the second digital model to identify the corresponding portion. In some examples, regenerating the second digital model includes associating the corresponding portion with a distinctive feature for display. In some examples, the operations include transmitting treatment data identifying the treatment target area of the organ to a radioablation treatment system.

In some examples, the operations include obtaining study data records for the patient, wherein each study data record identifies one of a plurality of study types and a study target area of a plurality of study target areas for studies performed on the patient. The operations also include determining a first number of each of the plurality of study types performed on the patient based on the study data records. Further, the operations include determining, for each of the plurality of study types, a second number of studies performed on the patient in each of the plurality of study target areas. The operation further include generating a first map for each of the plurality of study types based on the corresponding first number and second numbers. The operations also include storing the first map in the data repository. In some examples, each first map indicates a frequency of the corresponding study type on each of the plurality of study target areas. In some examples, the operations include generating a second map based on the first numbers and the second numbers, wherein the second map indicates a probability of treatment for each of the plurality of study target areas, and storing the second map in a data repository.

In some examples, a computer-implemented method includes a means for receiving a first input identifying an organ of a patient, and receive a scanned image of the organ. The method also includes a means for generating a first digital model of a type of the organ. Further, the method includes a means for determining an alignment of the scanned image to the first digital model. The method also includes a means for generating a second digital model comprising at least a portion of the scanned image and the first digital model. The method further includes a means for storing the second digital model in a data repository. In some examples, receiving the first input is in response to a selection of a portion of a displayed target definition map. In some examples, the organ is a heart. In some examples, the method includes a means for providing the second digital model for display.

In some examples, the method includes a means for receiving a second input identifying an adjustment to the alignment of the scanned image to the first digital model. The method also includes a means for adjusting the second digital model based on the second input. The method further includes a means for storing the adjusted second digital model in the data repository.

In some examples, the method includes a means for receiving a second input identifying a treatment target area of the organ. The method also includes a means for determining a corresponding portion of the second digital model based on the treatment target area of the organ. Further, the method includes a means for regenerating the second digital model to identify the corresponding portion. In some examples, regenerating the second digital model includes associating the corresponding portion with a distinctive feature for display. In some examples, the method includes a means for transmitting treatment data identifying the treatment target area of the organ to a radioablation treatment system.

In some examples, the method includes a means for obtaining study data records for the patient, wherein each study data record identifies one of a plurality of study types and a study target area of a plurality of study target areas for studies performed on the patient. The method also includes a means for determining a first number of each of the plurality of study types performed on the patient based on the study data records. Further, the method includes a means for determining, for each of the plurality of study types, a second number of studies performed on the patient in each of the plurality of study target areas. The method further includes a means for generating a first map for each of the plurality of study types based on the corresponding first number and second numbers. The method also includes a means for storing the first map in the data repository. In some examples, each first map indicates a frequency of the corresponding study type on each of the plurality of study target areas. In some examples, the method includes a means for generating a second map based on the first numbers and the second numbers, wherein the second map indicates a probability of treatment for each of the plurality of study target areas, and storing the second map in a data repository.

Although the methods described above are with reference to the illustrated flowcharts, it will be appreciated that many other ways of performing the acts associated with the methods can be used. For example, the order of some operations may be changed, and some of the operations described may be optional.

In addition, the methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A system comprising:
a computing device configured to:
receive a first input identifying an organ of a patient;
receive a scanned image of the organ;
generate a first digital model of a type of the organ;
determine an alignment of the scanned image to the first digital model;
generate a second digital model comprising at least a portion of the scanned image and the first digital model; and
store the second digital model in a data repository.

2. The system of claim 1, wherein the computing device is further configured to provide the second digital model for display.

3. The system of claim 1, wherein the computing device is further configured to:
receive a second input identifying an adjustment to the alignment of the scanned image to the first digital model;
adjust the second digital model based on the second input; and
store the adjusted second digital model in the data repository.

4. The system of claim 1, wherein the computing device is further configured to:
receive a second input identifying a treatment target area of the organ;
determine a corresponding portion of the second digital model based on the treatment target area of the organ; and
regenerate the second digital model to identify the corresponding portion.

5. The system of claim 4, wherein regenerating the second digital model comprises associating the corresponding portion with a distinctive feature for display.

6. The system of claim 4, wherein the computing device is further configured to transmit treatment data identifying the treatment target area of the organ to a radioablation treatment system.

7. The system of claim 1, wherein the computing device is further configured to:
obtain study data records for the patient, wherein each study data record identifies one of a plurality of study types and a study target area of a plurality of study target areas for studies performed on the patient;
determine a first number of each of the plurality of study types performed on the patient based on the study data records;
determine, for each of the plurality of study types, a second number of studies performed on the patient in each of the plurality of study target areas;
generate a first map for each of the plurality of study types based on the corresponding first number and second numbers; and
store the first map in the data repository.

8. The system of claim 7, wherein each first map indicates a frequency of the corresponding study type on each of the plurality of study target areas.

9. The system of claim 7, wherein the computing device is further configured to:
generate a second map based on the first numbers and the second numbers, wherein the second map indicates a probability of treatment for each of the plurality of study target areas; and
store the second map in the data repository.

10. The system of claim 9, wherein receiving the first input is in response to a selection of a portion of a displayed target definition map.

11. A computer-implemented method comprising:
receiving a first input identifying an organ of a patient;
receiving a scanned image of the organ;
generating a first digital model of a type of the organ;
determining an alignment of the scanned image to the first digital model;
generating a second digital model comprising at least a portion of the scanned image and the first digital model; and
storing the second digital model in a data repository.

12. The computer-implemented method of claim 11 comprising providing the second digital model for display.

13. The computer-implemented method of claim 11 comprising:
receiving a second input identifying an adjustment to the alignment of the scanned image to the first digital model;
adjusting the second digital model based on the second input; and
storing the adjusted second digital model in the data repository.

14. The computer-implemented method of claim 11 comprising:
receiving a second input identifying a treatment target area of the organ;
determining a corresponding portion of the second digital model based on the treatment target area of the organ; and
regenerating the second digital model to identify the corresponding portion.

15. The computer-implemented method of claim 14 comprising transmitting treatment data identifying the treatment target area of the organ to a radioablation treatment system.

16. The computer-implemented method of claim 11 comprising:
obtaining study data records for the patient, wherein each study data record identifies one of a plurality of study types and a study target area of a plurality of study target areas for studies performed on the patient;
determining a first number of each of the plurality of study types performed on the patient based on the study data records;
determining, for each of the plurality of study types, a second number of studies performed on the patient in each of the plurality of study target areas;
generating a first map for each of the plurality of study types based on the corresponding first number and second numbers; and
storing the first map in the data repository.

17. A non-transitory computer readable medium storing instructions that, when executed by at least one processor, cause the at least one processor to perform operations comprising:
receiving a first input identifying an organ of a patient;
receiving a scanned image of the organ;
generating a first digital model of a type of the organ;
determining an alignment of the scanned image to the first digital model;
generating a second digital model comprising at least a portion of the scanned image and the first digital model; and
storing the second digital model in a data repository.

18. The non-transitory computer readable medium of claim 17 wherein the operations further comprise:
receiving a second input identifying an adjustment to the alignment of the scanned image to the first digital model;
adjusting the second digital model based on the second input; and
storing the adjusted second digital model in the data repository.

19. The non-transitory computer readable medium of claim 17 wherein the operations further comprise:
receiving a second input identifying a treatment target area of the organ;
determining a corresponding portion of the second digital model based on the treatment target area of the organ; and
regenerating the second digital model to identify the corresponding portion.

20. The non-transitory computer readable medium of claim 17 wherein the operations further comprise:
obtaining study data records for the patient, wherein each study data record identifies one of a plurality of study types and a study target area of a plurality of study target areas for studies performed on the patient;
determining a first number of each of the plurality of study types performed on the patient based on the study data records;
determining, for each of the plurality of study types, a second number of studies performed on the patient in each of the plurality of study target areas;
generating a first map for each of the plurality of study types based on the corresponding first number and second numbers; and
storing the first map in the data repository.
